(19) United States
(12) Patent Application Publication
(10) Pub. No.: US 2009/0215669 A1
(43) Pub. Date: Aug. 27, 2009

(54) COMPOSITIONS AND METHODS FOR TREATMENT OF DIABETES

(75) Inventors: Mark A. Myers, Clayton (AU); Robyn Gray, Clayton (AU); Sarah Paule, Clayton (AU); Paul Zev Zimmet, Caulfield (AU)

Correspondence Address: SEED INTELLECTUAL PROPERTY LAW GROUP PLLC, 701 FIFTH AVE, SUITE 5400, SEATTLE, WA, 98104, US

(73) Assignee: DIA-B TECH LIMITED, Hawthorn (AU)

(21) Appl. No.: 12/094,693

(22) PCT Filed: Nov. 22, 2006
(86) PCT No.: PCT/AU06/01763

§ 371 Date: Oct. 27, 2008

(30) Foreign Application Priority Data
Nov. 22, 2005 (AU) 2005906489
May 11, 2006 (AU) 2006902486

(51) Int. Cl. A61K 38/28; C07K 14/62

(52) U.S. Cl. 514/4; 514/3; 530/303

(57) ABSTRACT

The present invention relates to a composition comprising a peptide of the formula: (Xaa)ₙ₁-Xaa₁-His-Thr-Asp-(Xaa)ₙ₂ wherein Xaa is any amino acid; Xaa₁ is a hydrophobic amino acid; n₁ is 0-10; and n₂ is 0-10; and derivatives thereof; and insulin. Complexes of insulin and the peptides, methods of dispersing multimeric insulin complexes and methods of regulating in vivo blood glucose levels, particularly in the treatment of diabetes are also described.

### FIELD OF THE INVENTION

The present invention relates to compositions comprising a class of hypoglycemic peptides and insulin. More particularly, the present invention relates to compositions of very fast acting insulin comprising insulin and a hypoglycemic peptide. Complexes of insulin and hypoglycemic peptides and methods of dispersing multimeric insulin complexes are also disclosed. The compositions containing hypoglycemic peptides and insulin have potential for use in control of diabetes particularly in diabetic subjects that require treatment with insulin.

### BACKGROUND OF THE INVENTION

The reference to any prior art in this specification is not, and should not be taken as, an acknowledgment or any form of suggestion that that prior art forms part of the common general knowledge in Australia.

Diabetes results in chronic hyperglycaemia due to the inability of the pancreas to produce adequate amounts of insulin or due to the inability of cells to utilise the insulin available. Many diabetic patients require treatment with insulin when the pancreas no longer produces insulin (Type 1 diabetes) or when inadequate amounts of insulin are produced by the pancreas (Type 2 diabetes).

Insulin is known to form hexameric complexes in the presence of zinc ions both in vivo and in vitro. However, insulin hexamers must dissociate into dimers or monomers before they can be absorbed and pass into the circulation and only insulin monomers bind to insulin receptors in the body. In order for insulin to be useful in the body, the hexameric complexes must disperse to provide insulin dimers or monomers. Dispersal of hexameric insulin complexes occurs naturally in the body but may take some time to occur delaying the onset of insulin activity. Since insulin is not absorbed and utilised in the body in hexameric form, it takes two to four hours from the time of administration of hexameric insulin preparations to achieve peak plasma insulin concentrations.

Insulin is available in three types, very fast acting insulin (eg Lispro™), fast acting also known as regular insulin and, intermediate acting or lente insulin. Often diabetic patients need a combination of shorter and longer acting insulin to ensure normal levels of blood glucose are maintained during the day, before and after meals, and during the long fasting period that occurs overnight.

Very fast acting insulin is used within 15 minutes before eating and can allow better control of blood sugar levels. It is easier to estimate time of eating within 15 minutes than within 30-60 minutes required for regular insulin. When using regular insulin a patient may eat too early or too late to provide the best blood glucose control. Another advantage of very fast acting insulin is reduced risk of hypoglycemia between meals.

Some very fast acting synthetic insulin analogues do not associate to form stable hexameric complexes, for example, NovoRapid™ and Lispro™. However, there is a need for improved very fast acting insulin products, particularly natural or synthetic insulin compositions.

Bioactive peptides have been described in WO 03/002594 as having hypoglycemic effects. The insulin-sensitising factor (ISF) Gly-His-Thr-Asp-NH₂ and its analogues have been prepared and shown to have insulin-sensitising activity (WO 03/002594).

In work leading to the present invention, ISF and its analogues have been found to disperse hexameric insulin complexes increasing the speed with which insulin can be absorbed and pass into the circulation and interact with its receptor in vivo. It has also been found that ISF is able to bind with insulin monomers to form an insulin-peptide complex, which may also assist in dispersing multimeric insulin complexes into monomeric form. Combinations of ISF and its analogues with insulin are therefore useful in treatment of diabetic patients that require insulin therapy, particularly when very fast acting insulin is required.

### SUMMARY OF THE INVENTION

In one aspect the invention provides a composition comprising a peptide of the formula:

(Xaa)ₙ₁-Xaa₁-His-Thr-Asp-(Xaa)ₙ₂

wherein
Xaa is any amino acid;
Xaa₁ is a hydrophobic amino acid;
n₁ is 0-10; and
n₂ is 0-10; and derivatives thereof;
   and insulin.

In another aspect the invention provides a composition comprising a peptide of the formula:

(Xaa)ₙ₁-Xaa₁-His-Thr-Asp-(Xaa)ₙ₂

wherein
Xaa is any amino acid;
Xaa₁ is a hydrophobic amino acid;
n₁ is 0-10; and
n₂ is 0-10; and derivatives thereof;
   and insulin;
   with the proviso that when Xaa₁ is Val, one of n₁ and n₂ is other than 0.

In yet another aspect of the invention, there is provided an insulin-peptide complex in which the insulin is associated with at least one peptide of the formula:

(Xaa)ₙ₁-Xaa₁-His-Thr-Asp-(Xaa)ₙ₂

wherein
Xaa is any amino acid;
Xaa₁ is a hydrophobic amino acid;
n₁ is 0-10; and
n₂ is 0-10; and derivatives thereof.

In some embodiments, the insulin-peptide complex has an insulin:peptide ratio of 1:1 or 2:1.

In preferred embodiments of the invention, the peptide is one of the formulae:

(Xaa)ₙ₁-Val-His-Thr-Asp-(Xaa)ₙ₂; or

(Xaa)ₙ₁-Gly-His-Thr-Asp-(Xaa)ₙ₂;

wherein Xaa, n₁ and n₂ are as defined above and derivatives thereof.

In some embodiments, the peptide in the composition is the tetrapeptide Gly-His-Thr-Asp (ISF401) or a C-terminal and/or N-terminal capped derivative thereof.

In yet another aspect of the invention there is provided a method of preparing a very fast acting insulin composition comprising the step of mixing a multimeric insulin complex with peptide of the formula:

(Xaa)ₙ₁-Xaa₁-His-Thr-Asp-(Xaa)ₙ₂

wherein
Xaa is any amino acid;
Xaa₁ is a hydrophobic amino acid;
n₁ is 0-10; and
n₂ is 0-10 and derivatives thereof.

In another aspect of the invention there is provided a method of dispersing multimeric insulin complexes comprising the step of exposing multimeric insulin complexes to a peptide of the formula:

(Xaa)ₙ₁-Xaa₁-His-Thr-Asp-(Xaa)ₙ₂

wherein
Xaa is any amino acid;
Xaa₁ is a hydrophobic amino acid;
n₁ is 0-10; and
n₂ is 0-10 and derivatives thereof.

In preferred embodiments, the multimeric insulin complexes are dimeric or hexameric complexes, especially hexameric complexes.

In a further aspect of the invention, there is provided a method of regulating in vivo blood glucose levels in a human or other mammal, which comprises administration of a combination comprising insulin and a peptide of the formula:

(Xaa)ₙ₁-Xaa₁-His-Thr-Asp-(Xaa)ₙ₂

wherein
Xaa is any amino acid;
Xaa₁ is a hydrophobic amino acid;
n₁ is 0-10; and
n₂ is 0-10 and derivatives thereof.

In yet a further aspect of the present invention, there is provided a method of treating diabetes in a human or other mammal comprising administration to said human or other animal a combination comprising insulin and a peptide of the formula:

(Xaa)ₙ₁-Xaa₁-His-Thr-Asp-(Xaa)ₙ₂

wherein
Xaa is any amino acid;
Xaa₁ is a hydrophobic amino acid;
n₁ is 0-10; and
n₂ is 0-10 and derivatives thereof.

In some embodiments, the diabetes is Type 1 diabetes. In other embodiments, the diabetes is Type 2 diabetes that requires administration of insulin.

In preferred embodiments of the methods of the invention the peptide is one of the formulae:

(Xaa)ₙ₁-Val-His-Thr-Asp-(Xaa)ₙ₂; or

(Xaa)ₙ₁-Gly-His-Thr-Asp-(Xaa)ₙ₂;

wherein Xaa, n₁ and n₂ are as defined above and derivatives thereof.

Preferably, the peptide is a tetrapeptide selected from
Val-His-Thr-Asp (ISF402); and
Gly-His-Thr-Asp (ISF401);
or C-terminal and/or N-terminal capped derivatives thereof.

In yet another aspect of the invention, there is provided a use of insulin and a peptide of the formula:

(Xaa)ₙ₁-Xaa₁-His-Thr-Asp-(Xaa)ₙ₂

wherein
Xaa is any amino acid;
Xaa₁ is a hydrophobic amino acid;
n₁ is 0-10; and
n₂ is 0-10; and derivatives thereof;
   in the manufacture of a medicament for treating diabetes in a human or other animal.

In some embodiments, the C-terminus and/or the N-terminus of the peptide used in the methods and compositions of the invention may be capped with a suitable capping group. For example, the C-terminus of the peptide may be amidated and/or the N-terminus of the peptide may be acylated, eg acetylated. In preferred embodiments, the C-terminus of the peptide is amidated.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### DESCRIPTION OF THE INVENTION

The present invention relates to a combination of insulin and a class of hypoglycemic peptides that may be used to provide very fast acting insulin in vivo. The hypoglycemic peptides may assist in dispersing multimeric insulin complexes to provide insulin that is readily absorbed into the circulation and is suitable for rapid binding to the insulin receptor. The hypoglycemic peptides may also have an insulin-sensitising effect thereby reducing insulin resistance. The combination of the invention is useful in treating diabetes that requires treatment with insulin, particularly in humans.

In one aspect the invention provides a composition comprising a peptide of the formula:

(Xaa)ₙ₁-Xaa₁-His-Thr-Asp-(Xaa)ₙ₂

wherein
Xaa is any amino acid;
Xaa₁ is a hydrophobic amino acid;
n₁ is 0-10; and
n₂ is 0-10; and derivatives thereof;
   and insulin.

Preferably, the peptide used is one of the formulae:

(Xaa)ₙ₁-Val-His-Thr-Asp-(Xaa)ₙ₂; or

(Xaa)ₙ₁-Gly-His-Thr-Asp-(Xaa)ₙ₂

wherein Xaa₁, n₁ and n₂ are as defined above and derivatives thereof.

In preferred embodiments, the peptide is a tetrapeptide selected from
Val-His-Thr-Asp (ISF402); and
Gly-His-Thr-Asp (ISF401).

In some embodiments of the composition a peptide in which Xaa₁ is Val and n₁ and n₂ are 0 is excluded.

In some embodiments and encompassed by the term “derivative”, the C-terminus of the peptide and/or the N-terminus of the peptide may be capped with a suitable capping group. For example, the C-terminus of the peptide may be amidated, and/or the N-terminus of the peptide may be acylated, eg. acetylated. In preferred embodiments, the C-terminus of the peptide is amidated.

As used herein, the term “amino acid” refers to compounds having an amino group and a carboxylic acid group. An amino acid may be a naturally occurring amino acid or non-naturally occurring amino acid and may be a proteogenic amino acid or a non-proteogenic amino acid. The amino acids incorporated into the amino acid sequences of the present invention may be L-amino acids, D-amino acids, α-amino acids, β-amino acids and/or mixtures thereof.

Suitable naturally occurring proteogenic amino acids are shown in Table 1 together with their one letter and three letter codes.

TABLE 1

| Amino Acid | one letter code | three letter code |
|---|---|---|
| L-alanine | A | Ala |
| L-arginine | R | Arg |
| L-asparagine | N | Asn |
| L-aspartic acid | D | Asp |
| L-cysteine | C | Cys |
| L-glutamine | Q | Gln |
| L-glutamic acid | E | Glu |
| glycine | G | Gly |
| L-histidine | H | His |
| L-isoleucine. | I | Ile |
| L-leucine | L | Leu |
| L-lysine | K | Lys |
| L-methionine | M | Met |
| L-phenylalanine | F | Phe |
| L-proline | P | Pro |
| L-serine | S | Ser |
| L-threonine | T | Thr |
| L-tryptophan | W | Trp |
| L-tyrosine | Y | Tyr |
| L-valine | V | Val |

Suitable non-proteogenic or non-naturally occurring amino acids may be prepared by side chain modification or by total synthesis. Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄. The amino group of lysine may also be derivatized by reaction with fatty acids, other amino acids or peptides or labeling groups by known methods of reacting amino groups with carboxylic acid groups.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulfhydryl groups may be modified by methods such as carboxymethylation with iodoacetic acid or iodoacetamide; performic acid oxidation to cysteic acid; formation of a mixed disulfides with other thiol compounds; reaction with maleimide, maleic anhydride or other substituted maleimide; formation of mercurial derivatives using 4-chloromercuribenzoate, 4-chloromercuriphenylsulfonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulfenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during protein synthesis include, but are not limited to, use of norleucine, 4-amino-butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. Examples of suitable non-proteogenic or non-naturally occurring amino acids contemplated herein is shown in Table 2.

TABLE 2

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisoleucine | Nmile |
| D-alanine | Dal | L-N-methylleucine | Nmleu |
| D-arginine | Darg | L-N-methyllysine | Nmlys |
| D-asparatic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl- -aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-naphthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-naphthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyl-naphthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanin | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl | Nmbc | | |
| ethylamino)cyclopropane | | | |

Suitable β-amino acids include, but are not limited to, L-β-homoalanine, L-β-homoarginine, L-β-homoasparagine, L-β-homoaspartic acid, L-β-homoglutamic acid, L-β-homoglutamine, L-β-homoisoleucine, L-β-homoleucine, L-β-homolysine, L-β-homomethionine, L-β-homophenylalanine, L-β-homoproline, L-β-homoserine, L-β-homothreonine, L-β-homotryptophan, L-β-homotyrosine, L-β-homovaline, 3-amino-phenylpropionic acid, 3-amino-chlorophenylbutyric acid, 3-amino-fluorophenylbutyric acid, 3-amino-bromophenyl butyric acid, 3-amino-nitrophenylbutyric acid, 3-amino-methylphenylbutyric acid, 3-amino-pentanoic acid, 2-amino-tetrahydroisoquinoline acetic acid, 3-amino-naphthyl-butyric acid, 3-amino-pentafluorophenyl-butyric acid, 3-amino-benzothienyl-butyric acid, 3-amino-dichlorophenyl-butyric acid, 3-amino-difluorophenyl-butyric acid, 3-amino-iodophenyl-butyric acid, 3-amino-trifluoromethylphenyl-butyric acid, 3-amino-cyanophenyl-butyric acid, 3-amino-thienyl-butyric acid, 3-amino-5-hexanoic acid, 3-amino-furyl-butyric acid, 3-amino-diphenyl-butyric acid, 3-amino-6-phenyl-5-hexanoic acid and 3-amino-hexanoic acid.

As used herein, the term “hydrophobic amino acid” refers to an amino acid with a hydrophobic side chain or no side chain. Suitable hydrophobic amino acids include, but are not limited to, glycine, L-alanine, L-valine, L-phenylalanine, L-isoleucine, L-leucine, L-methionine, L-tyrosine, D-valine, D-phenylalanine, D-isoleucine, D-leucine, D-methionine, D-tyrosine, L-β-homophenylalanine, L-β-homoisoleucine, L-β-homoleucine, L-β-homovaline, L-β-homomethionine, L-β-homotyrosine, cyclohexylalanine, L-norleucine and L-norvaline. Preferred hydrophobic amino acids are glycine, L-valine, L-phenylalanine, L-isoleucine and L-leucine, especially L-valine and glycine.

In some embodiments and encompassed by the term “derivative”, one or more of the His, Thr or Asp amino acids in the His-Thr-Asp sequence may be non-naturally occurring His, Thr or Asp. For example, the His, Thr or Asp may be D-amino acids or may be derivatised, for example by N-alkylation such as N-methylation or α-alkylation such as α-methylation. Examples of derivatised His, Thr and Asp include, but are not limited to, N-methyl-His, N-methyl-Thr, N-methyl-aspartic acid, α-methyl-histidine, α-methyl-threonine or α-methyl-aspartic acid. In preferred embodiments, the His, Thr and Asp are L-amino acids, and are underivatised.

Other derivatives include pharmaceutically acceptable salts. Examples of suitable salts include, but are not limited to, chloride, acetate, lactate and glutamate salts. Conventional procedures for preparing salts are known in the art.

The peptides incorporated in the compositions and complexes of the invention as described above may be synthesised using conventional liquid or solid phase synthesis techniques. For example, reference may be made to solution synthesis or solid phase synthesis as described in Chapter 9, entitled “Peptide Synthesis” by Atherton and Shephard, which is included in the publication entitled “Synthetic Vaccines” edited by Nicholson and published by Blackwell Scientific Publications. Preferably, a solid phase peptide synthesis technique using Fmoc chemistry is used, such as the Merrifield synthesis method (Wellings & Atherton (1997), In Methods in Enzymology, Vol 289, 44-66; Merrifield (1963), J. Am. Chem. Soc., 85, 2149).

Alternatively, these peptides may be prepared as recombinant peptides using standard recombinant DNA techniques. Thus, a recombinant expression vector containing a nucleic acid sequence encoding the peptide and one or more regulatory sequences operatively linked to the nucleic acid sequence to be expressed may be introduced into and expressed in a suitable prokaryotic or eukaryotic host cell, as described, for example, in Gene Expression Technology Methods in Enzymology, 185, Academic Press, San Diego, Calif. (1990), and Sambrook et al., Molecular Cloning: A Laboratory Manual, 2^{nd} ed. Cold Spring Harbor Laboratory Press, Cold Spring Harbor, N.Y. (1989).

The peptide Gly-His-Thr-Asp may also be isolated from human urine by standard protein purification procedures, preferably using reversed-phase high performance liquid chromatography (RP-HPLC). Using these procedures, Gly-His-Thr-Asp is obtained in isolated form. By “isolated” is meant a peptide material that is substantially or essentially freed from components, particularly other proteins and peptides, that normally accompany it in its native state in human urine by at least one purification or other processing step.

Such isolated peptide material may also be described as substantially pure. The term “substantially pure” as used herein describes peptide material that has been separated from components that naturally accompany it. Typically, peptide material is substantially pure when at least 70%, more preferably at least 80%, even more preferably at least 90%, and most preferably at least 95% or even 99% of the total peptide material (by volume, by wet or dry weight, or by mole percent or mole fraction) is the peptide of interest. Purity can be measured by any appropriate method, for example, in the case of peptide material, by chromatography, gel electrophoresis or HPLC analysis.

The insulin useful in the present invention may be of animal or human origin and may be synthetic or derivatised. Insulin of human origin may be identical to insulin produced by the human pancreas and may be synthetic or recombinant as known in the art. Alternatively, the insulin of human origin may be derivatised provided the derivatised insulin is capable of forming stable or unstable multimeric insulin complexes. Insulin of animal origin may be any of the insulin products of animal origin known in the art, such as those produced from pigs and cattle. In preferred embodiments the insulin is of human origin. The present invention may be useful with any type of insulin by enhancing formation or maintenance of monomeric insulin. Suitable forms of insulin include, but are not limited to Lantus™, Humulin UL™, Humulin 50/50™, Humulin L™, Humalog™, Humulin R™, Humulin NPH™, Humalog Mix 25™, Humulin 30/70™, Ultratard™, Monotard™, NovoRapid™, Actrapid™, Protaphane™, Novomix™, Mixtard 30/70™, Mixtard 50/50™, Mixtard 20/80™ and Levemir™.

While it is possible that, for use in therapy, the combination of peptide and insulin may be administered without other additives, it is preferable to present the combination together with one or more pharmaceutically acceptable carriers and/or diluents, and optionally other therapeutic and/or prophylactic agents. The carriers and/or diluents must be “acceptable” in the sense of being compatible with the other ingredients of the composition and not deleterious to the recipient.

As used herein, the term “combination of peptide and insulin” may refer to a composition comprising the peptide and insulin. In relation to methods of administration for therapy, the term “combination of peptide and insulin” includes administration of a composition of the invention and also includes separate administration of a composition containing the peptide and a composition containing insulin, either simultaneously or sequentially, such that the peptide and insulin interact with each other allowing dispersal of insulin multimers, in vivo after administration. In preferred embodiments, the combination of peptide and insulin are in one composition.

The formulation of such therapeutic compositions is well known to persons skilled in this field. Suitable pharmaceutically acceptable carriers and/or diluents include any and all conventional solvents, dispersion media, fillers, solid carriers, aqueous solutions, coatings, antibacterial and antifungal agents, isotonic and absorption delaying agents, and the like. The use of such media and agents for pharmaceutically active substances is well known in the art, and it is described, by way of example, in Remington's Pharmaceutical Sciences, 18th Edition, Mack Publishing Company, Pennsylvania, USA. Except insofar as any conventional media or agent is incompatible with the active ingredients, use thereof in the pharmaceutical compositions of the present invention is contemplated. Supplementary active ingredients can also be incorporated into the compositions.

It is especially advantageous to formulate such compositions in dosage unit form for ease of administration and uniformity of dosage. Dosage unit form as used herein refers to physically discrete units suited as unitary dosages for the human or other mammalian subjects to be treated; each unit contains a predetermined quantity of active ingredients calculated to produce the desired therapeutic effect in association with the required pharmaceutical carrier and/or diluent. The specifications for the novel dosage unit forms of the invention are dictated by and directly dependent on (a) the unique characteristics of the active ingredients and the particular therapeutic effect to be achieved, and (b) the limitations inherent in the art of compounding such an active ingredient for the particular treatment.

In another aspect of the invention, there is provided an insulin-peptide complex in which the insulin is associated with at least one peptide of the formula:

(Xaa)ₙ₁-Xaa₁-His-Thr-Asp-(Xaa)ₙ₂

wherein
Xaa is any amino acid;
Xaa₁ is a hydrophobic amino acid;
n₁ is 0-10; and
n₂ is 0-10; and derivatives thereof.

In some embodiments, the insulin-peptide complex has an insulin:peptide ratio of 1:1 or 2:1.

As used herein the term “associated with” when referring to the insulin-peptide complex means that the insulin and the at least one peptide are linked through peptide-peptide interactions such as hydrophilic or hydrophobic interactions, hydrogen bonding, ionic interactions or the bridging of polar or charged groups through metal ions.

In another aspect there is provided a method of preparing a very fast acting insulin composition comprising mixing a multimeric insulin complex with peptide of the formula:

(Xaa)ₙ₁-Xaa₁-His-Thr-Asp-(Xaa)ₙ₂

wherein
Xaa is any amino acid;
Xaa₁ is a hydrophobic amino acid;
n₁ is 0-10; and
n₂ is 0-10 and derivatives thereof.

As used herein, the term “very fast acting insulin composition” refers to an insulin composition that starts affecting blood glucose levels within one to 20 minutes after administration and provides a maximum or peak insulin level within one hour of administration. The very fast acting insulin composition provides blood glucose lowering effects for a duration of about 1 to 5 hours. Such fast acting insulin compositions are suitable for administration within about 15 minutes before eating.

As used herein the term “multimeric insulin complex” refers to a complex in which insulin molecules are associated with one another. In some embodiments the multimeric insulin complex is dimeric in which two insulin molecules are associated with one another. The insulin molecules may be associated by interactions such as hydrophobic or hydrophilic interactions, hydrogen bonding, ionic interactions or the bridging of polar or charged groups through metal ions. Another example of a multimeric insulin complex is a hexameric insulin complex in which six insulin molecules are associated with at least one metal ion in the II oxidation state. Insulin multimeric complexes may be formed with ions such as Zn(II), Co(II), Ni(II), Cu(II), Fe(II), Cd(II) and Pb(II) (Hill et al, Biochemistry, 1991, 30, 917-924. In preferred embodiments, the metal ion is Zn(II). It is known that under normal in vivo conditions, insulin is synthesised and stored in the pancreas until needed as stable hexameric complexes containing two Zinc(II) (Zn⁺⁺) ions. The hexameric complex also has a calcium (Ca(II)) binding site so calcium ions may also be present. Synthetic or recombinant insulin used in the treatment of diabetes also forms stable or unstable multimeric complexes.

In another aspect of the invention, there is provided a method of dispersing multimeric insulin complexes comprising the step of exposing multimeric insulin complexes to a peptide of the formula:

(Xaa)ₙ₁-Xaa₁-His-Thr-Asp-(Xaa)ₙ₂

wherein
Xaa is any amino acid;
Xaa₁ is a hydrophobic amino acid;
n₁ is 0-10; and
n₂ is 0-10 and derivatives thereof.

The term “exposing multimeric insulin complexes” includes allowing the peptide to come into contact with the multimeric insulin either in vitro or in vivo. Without wishing to be bound by theory, it is proposed that upon contact with multimeric insulin complexes, particularly hexameric complexes, the peptide binds the metal ions that are central to the complex destabilising the complex. Alternatively, the peptide may bind to an insulin molecule within the complex and destabilise the association between the insulin molecules. Contact between the multimeric insulin complex and the peptide may occur in vitro, for example during preparation of a very fast acting insulin composition. Alternatively, contact between the multimeric insulin complex and the peptide may occur in vivo, for example, after administration of separate compositions of multimeric insulin and peptide or when the peptide administered is allowed to act on endogenous hexameric insulin in vivo thereby correcting any deficiency in naturally occurring insulin-sensitising peptide (ISF). In one preferred embodiment, the multimeric insulin complex is exposed to the peptide before administration during the preparation of a very fast acting insulin composition. Preferably the insulin complex is a hexameric insulin complex.

In another embodiment of this aspect, there is provided a method of dispersing endogenous hexameric insulin complexes comprising the step of administering a peptide of the formula:

(Xaa)ₙ₁-Xaa₁-His-Thr-Asp-(Xaa)ₙ₂

wherein
Xaa is any amino acid;
Xaa₁ is a hydrophobic amino acid;
n₁ is 0-10; and
n₂ is 0-10 and derivatives thereof.

The present invention also extends to methods of regulating in vivo blood glucose levels in a human or other mammal by administering to the human or other mammal, a combination of peptide and insulin of the invention as described above.

As used herein, the term “human or other mammal” refers to humans and other warm blooded animals that may require regulation of blood glucose. For example, mammals includes domesticated animals such as dogs, cats, horses and the like, livestock animals such as cattle, sheep, pigs and the like, laboratory animals such as mice, rats, rabbits and the like, and captive animals such as those animals held in zoos. In a preferred embodiment, the subject is a human.

In this aspect of the present invention, without wanting to be bound by theory, the peptide is capable of exerting its effect by dispersing multimeric insulin complexes allowing rapid absorption of insulin into the circulation and rapid binding of monomeric insulin to insulin receptors. In addition to dispersing multimeric insulin complexes, in some embodiments the peptides may also provide an insulin-sensitising effect thereby reducing insulin resistance and allowing the monomeric insulin to be more effective. In some embodiments, the insulin-sensitising effects may be provided by an insulin-peptide complex.

In some aspects of the invention the methods of regulating in vivo blood glucose levels is used in a method of treating diabetes, in a human or other mammal. Type 1 diabetes is characterised by a requirement for treatment with insulin. Type 2 diabetes may require treatment with insulin if endogenous insulin production is too low to meet the needs of the patient.

In Type 1 diabetes there is a lack of insulin production. This is because the beta cells of the Islets of Langerhans in the pancreas have been destroyed, most often by autoimmune-mediated destruction. Those subjects with Type 1 diabetes require treatment with insulin to replace the insulin that would normally be produced in the pancreas. Since insulin is not produced, a subject with untreated or poorly controlled Type 1 diabetes will have hyperglycaemia.

In Type 2 diabetes, at least at the beginning of the disease, the pancreatic islet cells are capable of making large quantities of insulin. The transport of glucose across a cellular membrane is stimulated by insulin binding to its insulin receptor as part of an insulin signaling pathway. However, in Type 2 diabetes, the insulin signaling pathway malfunctions causing a condition called insulin resistance. Although there may be an abundance of insulin in the circulation, there is insufficient transport of glucose into cells and excess glucose production by the liver. This may cause not only hyperglycaemia but also hyperinsulinemia. As the disease progresses, there may be down-regulation of the insulin receptors and is some cases exhaustion of the beta cells. Once the beta cells are exhausted the amount of insulin produced may be too low or may stop and treatment with exogenous insulin may be required temporarily or possibly permanently to provide adequate insulin levels to control blood glucose levels.

The combination of the invention may be administered without other therapeutic agents or may be administered with, in a single composition, or separately, simultaneously or sequentially, with other therapeutic agents, for example, other forms of insulin or insulin-sensitising agents, provided that the other therapeutic agents do not affect the ability of the peptide to disperse multimeric insulin complexes. Examples of other forms of insulin include other forms of very fast acting insulin such as Lispro™ and Insulin Aspart™ (NovoRapid), fast acting such as Actrapid™, Hypurin Neutral™ and Humulin™, and intermediate acting insulin such as insulin glargine and lente insulin. Suitable insulin-sensitising agents include, but are not limited to, metformin (Glucophage™) and thiazolidinediones (also known as glitizones) such as Avandia™, (rosiglitazone) by GlaxoSmithKline and Actos™ (pioglitazone) by Takeda/Eli Lilly.

The combination of peptide and insulin may also reduce, prevent or slow the progression of complications associated with diabetes. Such complications include cardiovascular disease and associated complications such as diabetic dyslipidemia; high blood pressure (hypertension); neuropathy and nerve damage; kidney disease; and eye diseases such as glaucoma, cataracts and retinopathy.

A variety of administration routes are available. The particular mode selected will depend, of course, upon the particular condition being treated and the dosage required for therapeutic efficacy. The methods of this invention, generally speaking, may be practised using any mode of administration that is medically acceptable, meaning any mode that produces therapeutic levels of the active components of the invention without causing clinically unacceptable adverse effects. Such modes of administration include parenteral (e.g. subcutaneous, intramuscular and intravenous), oral, rectal, topical, nasal and transdermal routes. Preferably, the insulin or combination of insulin and peptide is administered by parenteral injection.

The active components may conveniently be presented in unit dosage form and suitable compositions for administration may be prepared by any of the methods well known in the art of pharmacy. Such methods include the step of bringing the active components into association with a carrier and/or diluent which may include one or more accessory ingredients. In general, the compositions are prepared by uniformly and intimately bringing the active components into association with a liquid carrier, a finely divided solid carrier, or both, and then, if necessary, shaping the product.

As will be appreciated by those skilled in the art, in the preparation of any formulation containing peptide actives, care should be taken to ensure that the activity of the peptide is not destroyed in the process and that the peptide is able to reach its site of action without being destroyed. In some cases, it may be possible to protect the peptide by means known in the art, such as microencapsulation. Similarly the route of administration should be chosen so that the peptide reaches its site of action.

Compositions suitable for parenteral administration conveniently comprise a sterile aqueous preparation of the active components which is preferably isotonic with the blood of the recipient. This aqueous preparation may be formulated according to known methods using those suitable dispersing or wetting agents and suspending agents. The sterile injectable preparation may also be a sterile injectable solution or suspension in a non-toxic parenterally-acceptable diluent or solvent, for example as a solution in polyethylene glycol and lactic acid. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid find use in the preparation of injectables.

Compositions of the present invention suitable for oral administration of a hypoglycemic peptide may be presented as discrete units such as capsules, cachets, tablets or lozenges, each containing a predetermined amount of the active component, in liposomes or as a suspension in an aqueous liquor or non-aqueous liquid such as a syrup, an elixir, or an emulsion.

Other delivery systems can include sustained release delivery systems. Preferred sustained release delivery systems are those which can provide for release of the active components of the invention in sustained release pellets or capsules. Many types of sustained release delivery systems are available; these include, but are not limited to: (a) erosional systems in which the active components are contained within a matrix, and (b) diffusional systems in which the active components permeate at a controlled rate through a polymer.

The combination of insulin and peptide may be delivered in well known devices used to deliver insulin. For example, the combinations of the invention may be delivered using insulin syringes, insulin delivery pens and insulin pumps.

The active components are administered in therapeutically effective amounts. A therapeutically effective amount means an amount necessary to at least partially control hyperglycaemia, or delay the onset of hyperglycaemia. Such amounts will depend on the type of hyperglycaemia or diabetes being treated, the severity of the condition, the individual patient parameters such as age, physical condition, size, weight, extent of insulin resistance and concurrent treatment, and the timing of the therapy, for example, immediately before a meal or at the time of a severe hyperglycemic event. A typical daily dose of insulin used by a Type 1 diabetic is in the range of 0.1 to 2.5 units/kg, more typically 0.5 to 1 unit/kg/day. In Type 2 diabetes, a starting dose of insulin for augmentation therapy is 0.15 units/kg/day, with therapy often being in the range of up to 15 to 20 units per day. When insulin is administered in combination with a peptide, a lower dose of insulin may be utilised. A person skilled in the art, such as an attending physician may determine suitable amounts of insulin and peptide by monitoring the blood glucose of a patient after administration and food intake.

Generally, daily doses of hyperglycemic peptide will be from about 0.01 mg/kg per day to 1000 mg/kg per day. Small doses (0.01-1 mg) may be administered initially, followed by increasing doses up to about 1000 mg/kg per day. In the event that the response in a subject is insufficient at such doses, even higher doses (or effective higher doses by a different, more localised delivery route) may be employed to the extent patient tolerance permits. Multiple does per day are contemplated to achieve appropriate systemic levels of hypoglycemic peptide.

In some embodiments, the ratio of peptide molecule to insulin molecule is in the range of 1:6 to 6:1, preferably 2:6 to 5:1, 2:6 to 4:1, 2:6 to 3:1, 2:6 to 2:1 or 2:6 to 1:1. In other embodiments, the ratio of peptide to insulin is 0.5 mg to 5 mg peptide per unit of insulin, especially about 1.5 to 4 mg peptide to 1 unit of insulin, more especially about 3 mg peptide to 1 unit of insulin.

Further features of the present invention are more fully described in the following Example(s). It is to be understood, however, that this detailed description is included solely for the purposes of exemplifying the present invention, and should not be understood in any way as a restriction on the broad description of the invention as set out above.

### BRIEF DESCRIPTION OF THE DRAWINGS

FIG. 1A graphically represents a time-course of blood glucose levels in female Zucker fa/fa rats injected with a combination of insulin (1 unit/kg body weight) and ISF401 (1.5 mg/kg) (—♦—) or insulin in the absence of ISF401 (—□—).

FIG. 1B graphically represents a time-course of insulin levels in female Zucker fa/fa rats injected with a combination of insulin (1 unit/kg body weight) and ISF401 (1.5 mg/kg) (—♦—) or insulin in the absence of ISF401 (1.5 mg/kg) (—□—).

FIG. 1C graphically represents a time-course of C-peptide levels in female Zucker fa/fa rats injected with a combination of insulin (1 unit/kg body weight) and ISF401 (1.5 mg/kg) (—♦—) or insulin in the absence of ISF401 (—□—).

FIG. 2 graphically represents dose-response curve for ISF402 injected intravenously with or without insulin in female Zucker fa/fa rats. Insulin (black bars) was injected alone (n=11) or with 0.5 (n=7), 1.5 (n=6), 3 (n=8) or 4.5 mg/kg (n=6) ISF402, or ISF402 was injected alone (white bars) at doses of 0 (n=5), 3 (n=6), 4.5 (n=6) or 10 mg/kg (n=7). The AUC for the (A) Relative blood glucose concentration, (B) Serum insulin concentration and (C) Relative serum C-peptide concentration over 60 minutes after injection. The error bars represent standard error of the mean, an unpaired t test was used for comparison. ^{∥}P=0.0009 for insulin+ISF402 (0.5 mg/kg) compared to insulin, *P<0.005 for insulin+ISF402 (1.5 mg/kg) compared to insulin, ^{†}P<0.05 for insulin+ISF402 (3 mg/kg) compared to insulin, ^{‡}P<0.05 for insulin+ISF402 (4.5 mg/kg) compared to insulin. ^{§}P<0.03, ISF402 (4.5 mg/kg) compared to saline.

FIG. 3 graphically represents the extracellular acidification rate (ECAR) in C2C12 cells upon exposure to insulin at various concentrations, alone (——) or in combination with 0.1 μM ISF402 (—▪—).

FIG. 4A graphically represents a time-course of insulin levels in female Zucker fa/fa rats injected with a combination of insulin (1 unit/kg body weight) and ISF402 (1.5 mg/kg) (—▪—) or insulin in the absence of ISF402 (—▴—).

FIG. 4B graphically represents a time-course of serum C-peptide levels in female Zucker fa/fa rats injected with a combination of insulin (1 unit/kg body weight) and ISF402 (1.5 mg/kg) (—▪—) or insulin in the absence of ISF402 (—▴—).

FIG. 5 provides a CD spectrum of Zn-insulin (1 mg/mL in 25 mM Hepes buffer, before (black line) and after (grey line) addition of EDTA.

FIG. 6 graphically represents the change in CD signal of Zn-insulin (1 mg/mL tris buffer, pH 7.4) at 275 nm upon addition of 1 μL quantities of 100 mM ISF401 (white bars), 4 mM EDTA (black bars) or deionised water (hatched bars).

FIG. 7 provides a graphical representation of the elution volumes of insulin (solid line), ISF402 (dotted line) and a mixture of ISF402 and insulin (dashed line) during gel filtration chromatography.

FIG. 8 is a photographic representation showing ISF401 is colocalised with insulin in pancreatic islets of mouse and human. A rabbit antiserum was raised to ISF401 conjugated to diphtheria toxoid. Tissue sections were blocked and incubated with the anti-ISF401 antibody and an anti-insulin (Dako) antibody. Sections were further incubated with secondary antibodies to insulin anti-rabbit IgG conjugated to Alex568 (Molecular probes), and ISF401, anti-guinea pig IgG conjugated to FITC (Dako). (A) Mouse pancreas. Anti-ISF401 (red) bound to the same cells as anti-insulin (green) indicating colocalisation (orange/yellow) with insulin in pancreatic islet beta cells. (B) Human pancreas from a male donor age 77 showing a small cluster of insulin positive islet beta cells. (C) Human pancreas showing an islet with a central ductule (Dako). (D) MIN6 mouse islet beta cells. Scale bar for (A-C) is 50 μm and for (D) 25 μm.

FIG. 9 is a reverse phase HPLC trace. ISF401 is secreted by MIN6 cells. Serum free media conditioned for 24 hours by a confluent layer of MIN6 cells was separated by reverse phase HPLC. Absorption at 214 nm for the conditioned media (solid line) and unconditioned serum free media (dashed line) identified the appearance of several peaks in conditioned media, including a species with a retention time equivalent to synthetic ISF401 (16.2 minutes, arrowed). This species was identified as ISF401 by MALDI-TOF mass spectrometry.

FIG. 10 is a graphical representation showing urinary excretion of ISF401 increases after feeding. The amount of ISF401 secreted per hour (nanogram per hour) over 12 or 24 hours was measured by HPLC. The identity of the peptide peak was verified by MALDI-TOF mass spectrometry for selected samples. Lean and obese (fa/fa) Zucker rats were fasted for 24 hr (fasted) with urine collection over the last 12 hours, then provided with feed and urine collected for a further 12 hours (fed). The difference between lean fasted and lean fed was significant (P=0.008, Mann-Whitney U-test). Urinary ISF401 did not increase in two of the five obese rats.

FIG. 11 graphically represents the full time course for blood glucose, insulin and C-peptide for the optimum ISF402 doses shown in FIG. 2. Rats were intravenously injected with insulin alone (n=11) (open square) or insulin+ISF402 at 1.5 mg/kg (n=6) (closed square). (A) Relative blood glucose concentration, (B) serum insulin concentration and (C) Relative serum C-peptide concentration. Another group of rats were intravenously administered saline (n=5) (open circle) or ISF402 at 4.5 mg/kg (n=6) (closed circle). (D) Relative blood glucose concentration, (E) serum insulin concentration and (F) Relative serum C-peptide concentration. The error bars represent standard error of the mean, an unpaired t test was used for comparison. *P<0.05 for insulin (1 U/kg)+ISF402 (1.5 mg/kg) compared to insulin. ^{†}P<0.05 for ISF402 (4.5 mg/kg) compared to saline.

FIG. 12 graphically indicates that insulin clearance was not altered by injection of ISF402 at 4.5 mg/kg in 16-20 week old female Zucker fa/fa rats. Insulin clearance was derived by dividing the area under the curve for the molar C-peptide by the area under the curve for the molar insulin for each rat. Rats were intravenously administered with saline (n=5) (white bar) or 4.5 mg/kg of ISF402 (n=6) (black bar). The error bars represent standard error of the mean, an unpaired t test was used for comparison.

FIG. 13 graphically represents the effects on blood glucose concentration, serum insulin concentration and serum C-peptide concentration in male Zucker fa/fa rats intravenously injected with insulin and ISF402 at 1.5 mg/kg for 60 minutes. Insulin was injected alone (n=5) (open square) or with 1.5 mg/kg of ISF402 (n=8) (closed square). (A) Relative blood glucose concentration, (B) serum insulin concentration and (C) circulating serum C-peptide concentration. The error bars represent standard error of the mean, an unpaired t test was used for comparison. *P=0.01 for Insulin (1 U/kg)+ISF402 (1.5 mg/kg) compared to Insulin (1 U/kg).

FIG. 14 graphically represents the effects of intravenous injection of Lispro insulin (1 U/kg) and Lispro insulin with ISF402 (1.5 mg/kg) in female Zucker rats. (A) Relative blood glucose, (B) Relative C-peptide concentration for female Zucker rats treated with Lispro insulin (white bar) and Lispro insulin with ISF402 (black bar). Each treatment group had more than 5 rats. The error bars represent standard error of the mean, an unpaired t test was used for comparison.

FIG. 15 provides a graphical representation of the solubility of ISF402 at various pH and temperatures. The samples at room temperature (23-25° C.) and 37° C. were incubated for 24 hours and analyzed by HPLC and UV (214 nm) absorption to determine the concentration of ISF402.

FIG. 16 provides representations demonstrating the stability of BSA and ISF402 in simulated gastric and intestinal fluids. Non reducing SDS-gel electrophoresis of bovine serum albumin digested with simulated gastric fluid (A) and simulated intestinal fluid (B) over 300 minutes. ISF402 was incubated with stimulated gastric or intestinal fluid and (C) the retention time by C18 reverse phase HPLC and (D) area under the curve at 214 nm absorbance in μVxsec were used to quantitate the remaining peptide.

FIG. 17 provides mass spectra confirming the structural integrity of ISF402 after 7 hours incubation in simulated gastric and intestinal fluid by ESI-MS. ISF402 remains intact with a molecular weight of 470 Da (circled) after 420 minutes in (A.) SGF, (B.) SIF. (C.) Lower molecular species in the spectrum are present in the buffer blank.

FIG. 18 provides graphical representations of the results of intraperitoneal injection of insulin (2 U/kg) with ISF402 (3.0 mg/kg) in female Zucker fa/fa rats. The (A) relative blood glucose, (B) insulin concentration and (C) relative C-peptide concentration for female Zucker fa/fa rats were treated with ISF402 (n=6) and/or insulin alone (n=11) were determined for 120 minutes. The area under the curve (AUC) for relative (D) blood glucose, (E) serum insulin concentration and (F) circulating C-peptide concentration were calculated over 120 minutes for rats intraperitoneally administered with insulin (2 U/kg)±ISF402 at 3.0 mg/kg. The error bars represent standard error of the mean, an unpaired t test was used for comparison. P=0.006, 0.002, 0.015, at the time point with largest difference between treated and control groups for relative blood glucose, serum insulin and serum c-peptide respectively.

FIG. 19 provides graphical representations showing the effect of repeated and single oral dose of ISF402 on insulin sensitivity. 16-20 week old female Zucker fa/fa rats were given ISF402 orally at 15 mg/kg followed by an introperitoneal insulin tolerance test and three days later were given 30 mg/kg ISF402 or saline followed by an intraperitoneal insulin tolerance test. A second study group of rats were given a single dose of ISF402 30 mg/kg or saline followed by an intraperitoneal insulin tolerance test. The AUC for the (A) relative blood glucose for consecutive oral administration of ISF402 at 15 mg/kg and 30 mg/kg was determined after 150 minutes of ISF402 treatment. The AUC for the relative (B) blood glucose (C) insulin and (D) C-peptide were determined after 150 minutes for a single oral dose of ISF402 at 30 mg/kg. The error bars represent standard error of the mean, an unpaired t test was used for comparison. ^{†}P=0.04 versus multiple dose control. *P=0.02 versus single dose control (n=5).

FIG. 20 provides graphical representations showing the effects of administration of ¹⁴C-ISF402 in 16-20 week old female Zucker fa/fa rats. Sera and whole blood from rats intravenously injected with 1.98-2.06 μCi/kg of ¹⁴C-ISF402 at a final concentration of 4.5 mg/kg body weight ISF402 (A) or orally administered with 5.0-5.1 μCi/kg of ¹⁴C-ISF402 at a final concentration of 30 mg/kg body weight ISF402 (B). Serum and blood samples were collected over 120 minutes and 4 hours respectively. n=3 for each group.

FIG. 21 provides graphical representations allowing determination of whether the ¹⁴C-ISF402 remained intact in serum and urine samples by RP-HPLC analysis. Analysis of ¹⁴C-ISF402 was undertaken on (A) ¹⁴C-ISF402 that was administered to the rats, (B) ¹⁴C-ISF402 mixed with ISF402 to a final concentration of 30 mg/ml before oral administration to the rats, (C) a serum sample collected 2 minutes after intravenous injection of ¹⁴C-ISF402, (D) a serum sample 120 minutes after oral administration of ¹⁴C-ISF402, (E) a urine sample collected during the 4 hours after oral administration and (F) a urine sample collected during the 12 hours after oral administration.

FIG. 22 provides a graphical representation of competitive Inhibition binding curves for ISF401 diluted in casein (O). (A) Free ISF (6.1 pg/ml to 50 μg/ml) was incubated with casein. (B) The linear range of detection of free ISF is 97 pg/ml to 6.25 μg/ml. Both curves indicate percentage of inhibition versus concentration of inhibitor peptide. The points were the mean±SEM from five or more experiments.

FIG. 23 provides a graphical representation of the correlation between the immunoassay and the HPLC method for determining ISF in urine from various rat samples. A scatter plot of ISF levels determined by the two methods is shown. The solid line is the line of identity. The correlation co-efficient R²=0.92.

FIG. 24 graphically demonstrates the dissociation of insulin hexamers in the presence of ISF401 (GHTD-amide). Size exclusion chromatography with UV monitoring at 214 nm shows hexameric insulin elutes as a single peak at 13.315 mL (FIG. 24A, solid line). Incubation of insulin with ISF401 followed by size exclusion chromatography with UV monitoring at 276 nm indicates a reduction in the amount of hexameric insulin as shown by broadening of the peak at 13.39 to 13.95 mL and the emergence of peaks at 15.085 mL and 19.045 mL corresponding to dimeric insulin (11.8 kDa) and monomeric insulin (5.8 kDa) respectively (FIG. 24B, broken line). Incubation of insulin with a control tetrapeptide NCP, which does not chelate zinc ions, followed by size exclusion chromatography with UV monitoring at 214 nm showed the presence of two distinct peaks, (13.21-14.09 mL, hexameric insulin and 18.56 mL, NCP) (FIG. 24C, broken line). Monitoring of the elution of insulin and NCP at 276 nm showed that there is no hexameric insulin in the NCP peak at 18.56 minutes (FIG. 24D).

### EXAMPLES

### Peptides

ISF401 (Gly-His-Thr-Asp-NH₂) and ISF402 (Val-His-Thr-Asp-NH₂) were synthesised by standard protein synthetic methods using Fmoc chemistry. Peptides were >95% pure as determined by reverse phase high performance liquid chromatography (RP-HPLC).

Insulin

Insulin (Bovine pancreas) containing 0.6% zinc was purchased from Sigma. The sodium salt of insulin (zinc-free insulin) was bovine insulin purchased from Calbiochem. Lispro (Humalog) was obtained from Eli Lilly (Eli Lilly, NSW. Australia).

Circular Dichroism (CD)

Zinc-insulin hexamers were prepared by dissolving bovine insulin containing 0.6% zinc in 6M HCl then raising the pH to 7.2-7.4 by addition of NaOH. HEPES buffer was added to give a final solution of 1 mg/mL insulin in 25 mM HEPES buffer (pH 7.2). The CD spectrum of insulin or ISF401 was measured from 190-250 nm at 20° C. on a Jasco J-810 spectropolarimeter equipped with a PFD 423S/L Peltier type temperature controller. 200 μL of sample was placed in a quartz cuvette, with path length of 1 mm, in the spectropolarimeter and the CD spectrum was recorded. Each spectrum represents an average of 3-5 scans performed at 100 nm/min with a band width of 1 nm. The effect of ethylenediamine tetraacetic acid (EDTA) and ISF401 were measured by adding an aliquot of EDTA (4 mM) or an aliquot of ISF401 (100 mM) and measuring the CD spectrum.

Intravenous Injection of Zucker fa/fa Rats

The Monash University Animal Ethics Committee approved all procedures performed on experimental animals. Zucker rats were purchased from the Monash University Central Animal Facility. Insulin and ISF401 were injected through the femoral vein while the rat was under anaesthesia (Pentobarbitone) and rats were humanely killed while still unconscious at the end of the procedure. The insulin concentration used for all in vivo experiments was 1 unit per kg of body weight. Blood was collected from the tail vein and glucose concentrations were measured using a Medisense glucometer. Insulin and C-peptide concentrations were measured in serum samples using a Linco Rat Insulin RIA kit according to the manufacturers instructions. Group sizes were between 5 and 8 rats per treatment.

Microphysiometry

C2C12 mouse myotube cells were seeded onto supports and differentiated. The cells were then placed in a Cytosensor (Molecular Devices) in non-buffered pH sensitive RPMI 1640 media. Once the cells were equilibrated at 37° C. with RPMI 1640 media (zero control), ISF402, insulin or ZnCl₂ at increasing concentrations were added for a duration of 20 minutes, for each treatment. The Cytosensor measured the changed in pH as a response of cells to the treatment.

### Example 1

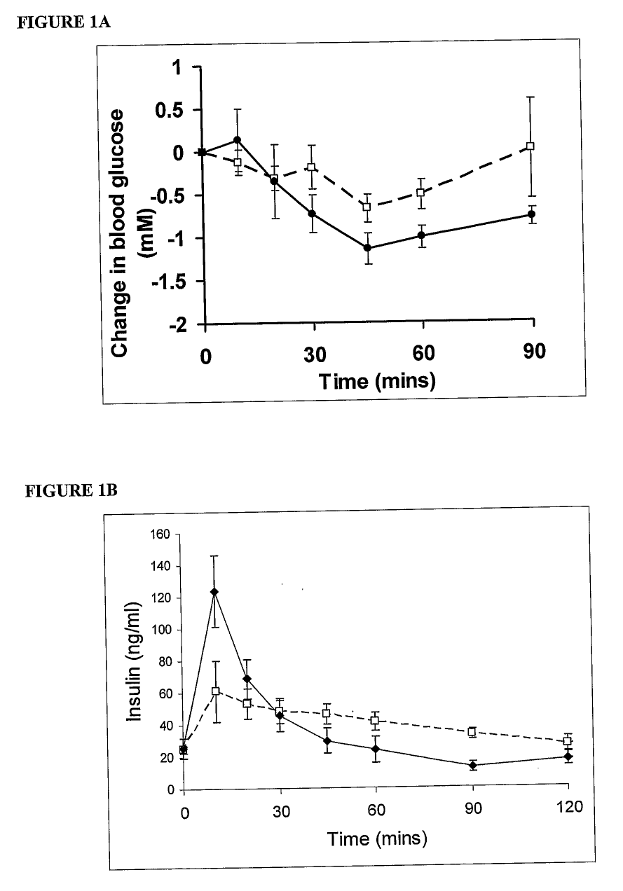

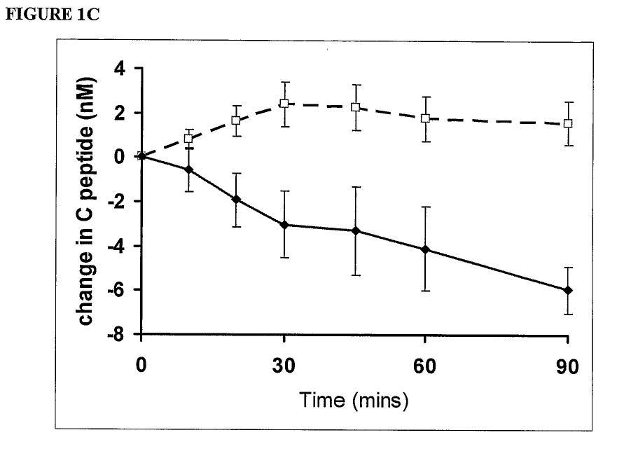

Female Zucker fa/fa rats were injected with ISF401 at varying doses either with insulin at 1 unit/kg body weight or without insulin. Blood glucose was measured on a drop of tail vein blood using a medisense glucometer (Abbott). Blood glucose measurements were performed and serum was collected at various times after injection and serum insulin and C-peptide measured using Linco RIA kits. A significant reduction in blood glucose was observed 30 to 90 minutes after injection when compared with controls injected with insulin alone (FIG. 1A). There was a significant increase in serum insulin concentration after injection with ISF401 and insulin with a peak insulin concentration at 10 minutes post injection. The peak insulin levels were significantly greater than those observed when insulin was injected alone (FIG. 1B).

The increase in peak insulin levels was not due to secretion of endogenous insulin from the pancreas as shown by the lack of an increase in serum C-peptide concentration after ISF401 injection. Instead, C-peptide concentration decreased, reflecting a reduced requirement for pancreatic insulin secretion in response to increased insulin sensitivity induced by ISF401 (FIG. 1C).

### Example 2

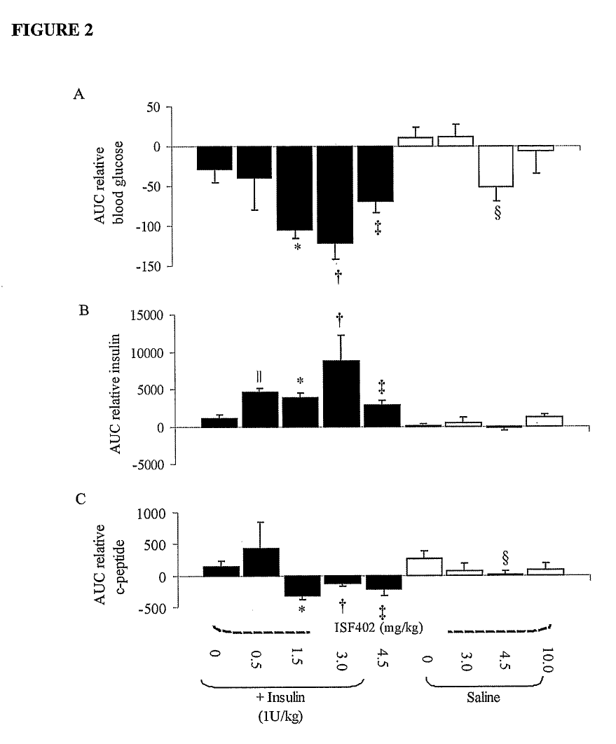

The effect of ISF402 dose on glucose homeostasis was tested both with and without simultaneous injection of exogenous insulin. Doses of 0.5, 1.5, 3 and 4.5 mg/kg of ISF402 with insulin and 3, 4.5 and 10 mg/kg of ISF402 alone were injected intravenously into the femoral vein of female Zucker rats and blood glucose, C-peptide and insulin were measured as before. Across the range of ISF402 doses, whether with or without co-injection of exogenous insulin, the glucose lowering response was dose dependent and bell shaped (FIG. 2A). When ISF402 was co-injected with exogenous insulin the decrease in blood glucose concentration was dose-dependent and inversely correlated with serum insulin levels. At the same time, endogenous insulin production was reduced as shown by a decrease in C-peptide (FIGS. 2B and 2C). ISF402 without insulin also lowered blood glucose but only at a dose of 4.5 mg/kg. In this case there was no observed increase in circulating insulin but serum C-peptide concentrations were reduced (FIGS. 2A and B) indicating that ISF402 is not stimulating insulin secretion.

### Example 3

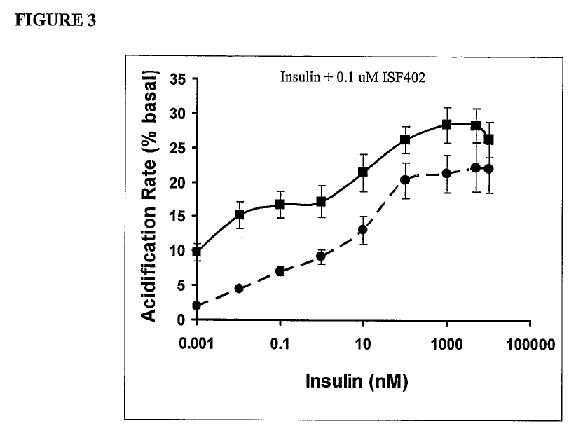

The activity of the combination of insulin and ISF402 in insulin sensitisation was explored in C2C12 muscle cells using microphysiometry, a technique that measures extracellular acidification as an indicator of cell metabolism. An ISF402 concentration of 0.1 μM was used to test for sensitisation of insulin responsiveness as this concentration produces a sub-maximal (˜20%) response in C2C12 cells. In the presence of 0.1 μM ISF402, the cellular response to insulin was increased as shown by increasing extracellular acidification rate (ECAR) with increasing concentrations of insulin, particularly at low insulin concentrations (FIG. 3).

### Example 4

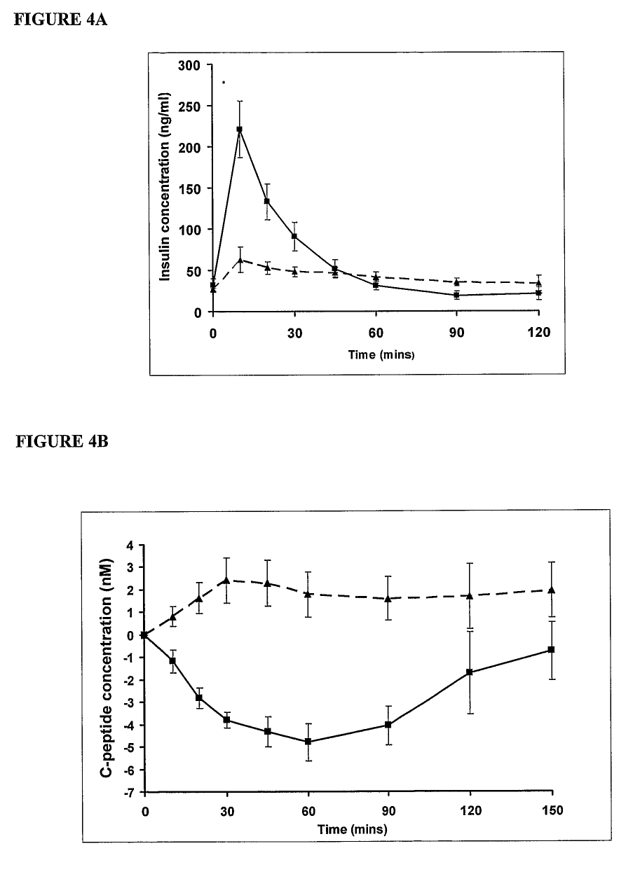

Female Zucker fa/fa rats were injected with ISF402 at 1.5 mg/kg and 1 unit/kg insulin or 1 unit/kg insulin alone. Serum was collected at various times and serum insulin was measured. There was a significant increase in serum insulin concentration after injection with ISF402 and insulin with a peak insulin concentration at 10 minutes post-injection. The peak insulin levels were significantly greater than those observed when insulin was injected alone (FIG. 4A).

The increase in peak insulin levels was not due to secretion of endogenous insulin from the pancreas as shown by lack of an increase in serum C-peptide concentration after ISF402 injection (FIG. 4B). Instead, C-peptide concentration decreased, reflecting a reduced requirement for pancreatic insulin secretion in response to increased insulin sensitivity induced by ISF402.

### Example 5

Insulin readily forms hexamers that are co-ordinated and stabilised by two Zn²⁺ ions. Insulin binds to its receptor as a monomer, hence hexameric insulin must dissociate into monomeric form to be biologically active. The release of subcutaneously injected insulin is usually slow due to the requirement for hexamer dissociation to occur before entry into the blood stream. One explanation for the effect of ISF peptides on the peak of serum insulin levels after intravenous injection (see FIG. 1) is that ISF peptides speed the dispersion of hexameric insulin so producing a sharp peak of free insulin in the circulation.

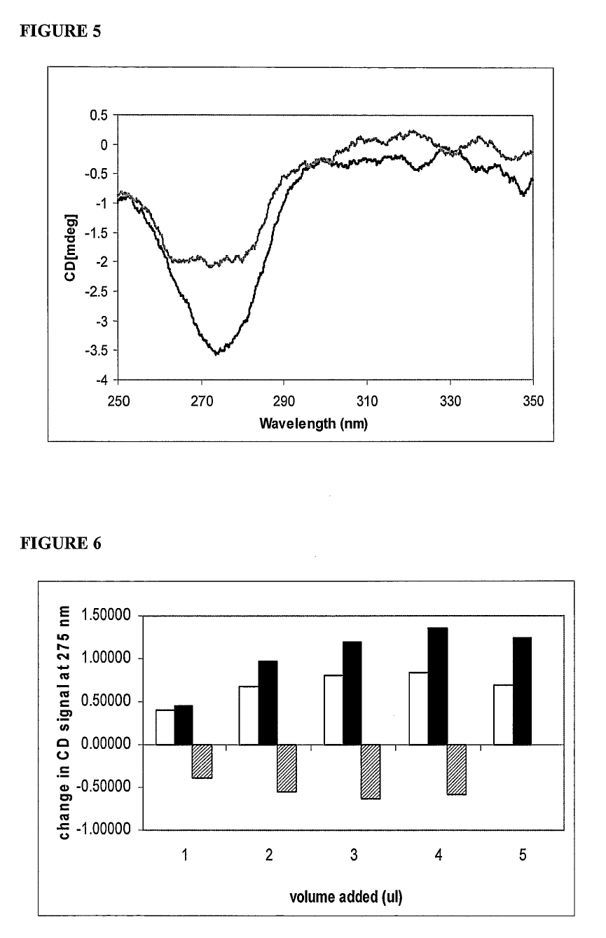

To test dispersal of insulin hexamers by ISF401, circular dichroism (CD) was used. The CD profile for hexameric insulin (Zn-insulin, 1 mg/mL in 25 mM HEPES buffer, pH 7.2) has a strong negative peak at 275 nm (FIG. 5). Addition of the transition metal chelator EDTA at a concentration of 2.5 mM, which binds to the zinc allowing the insulin hexamers to disperse and form monomeric/dimeric insulin, leads to an increase in the signal at 275 nm (FIG. 5).

The CD signal at 275 nm was used to measure the association state of insulin in the presence of ISF401 (1 μL of 100 mM), EDTA (1 μM of 4 mM) and deionised water (1 μL). The CD signal increased upon addition of ISF401 or EDTA consistent with the dispersal of insulin hexamers (FIG. 6). The amount of ISF401 required for maximum dispersion of insulin hexamers was between 1.5 and 2 mM, which is a 10 fold molar excess to insulin.

### Example 6

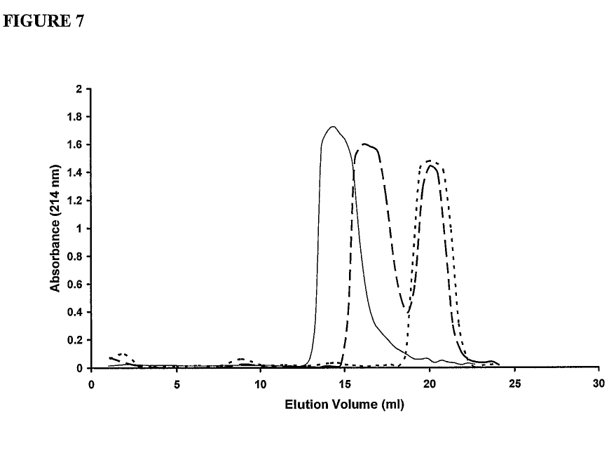

The effect of ISF402 on the multimerisation of insulin was examined in vitro by mixing insulin with ISF402 and separating molecular complexes by gel filtration chromatography, which separates on the basis of size (FIG. 7). Comparison of the retention volume of a mixture of ISF402 and insulin (dashed line) with either of insulin (solid line) or ISF402 alone (dotted line) showed that insulin eluted from the column later when mixed with ISF402 (16-17 mL) than when eluted without pre-mixing with ISF402 (14-15 mL). This suggests that ISF402 reduces the size of the insulin multimer.

### Example 7

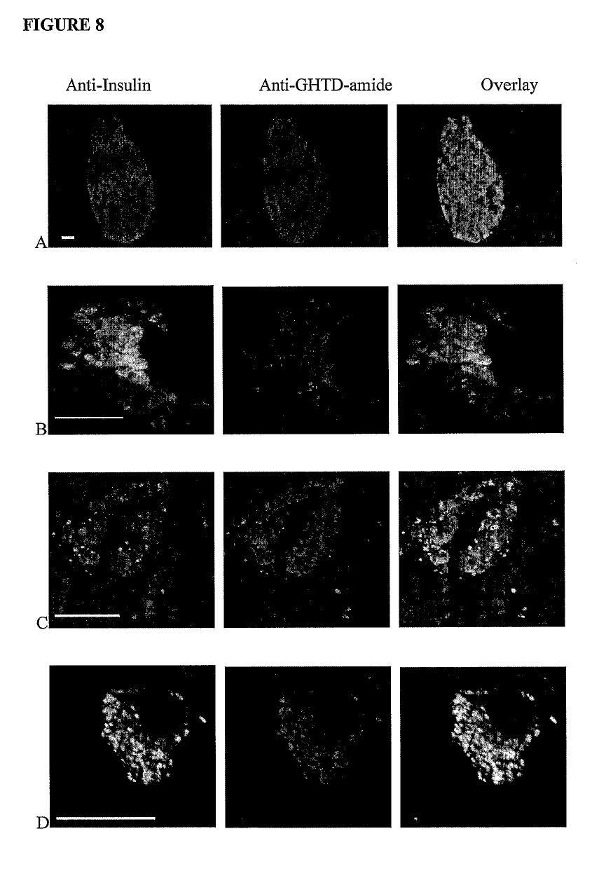

Peptides in urine usually derive from fragmented plasma proteins or bioactive peptides normally present in the circulation (Cutillas et al., Clinical Science, 104:483-490 (2003)). Carboxyl terminal amidation is a feature usually associated with neuropeptides and peptide hormones suggesting that ISF401 may be a circulating peptide hormone or hormone fragment with a natural role in increasing insulin sensitivity. To identify the source of urinary ISF401 an antiserum to ISF401 was raised by conjugation of the amino terminus of ISF401 to diphtheria toxoid and immunisation of rabbits. Indirect immunofluorescence identified strong co-localisation of the anti-serum with insulin in the pancreatic islets of Langerhans of both mouse (FIG. 8A) and human (FIGS. 8B and C). Mouse liver, muscle, kidney and adipose tissues and other endocrine glands from human were all negative (data not shown). Specificity of antibody binding was confirmed by lack of staining of pre-immune serum, lack of cross reaction of the second antibodies, and inhibition of staining by addition of synthetic ISF401 peptide. Confocal imaging of mouse islet beta cell-derived MIN6 cells revealed co-localisation of anti-ISF401 with insulin secretory granules (FIG. 8D).

### Example 8

### Methods

Urine was collected from lean (n=5) and fa/fa Zucker rats (n=5) as follows. Rats were fasted for 12 hours then placed in metabolic cages. Urine was then collected for a 12 hour period (fasted), after which standard rat chow was provided and urine collected for a further 12 hours (fasted-fed).

MIN6 cells were cultured at 37 degrees Celsius, 5% CO₂ in DMEM with 10% FCS. For analysis of peptide secretion, a near confluent layer of cells were washed then incubated in serum free media for 24 hours.

ISF401 in urine and cell culture media was detected by HPLC chromatography. Samples were centrifuged at 13000×g for 5 minutes before loading onto a Phenomenex Luna(2), 4.6×150 mm C-18 column that had been equilibrated with 10% buffer B (90% acetonitrile, 0.1% v/v H₃PO₄, 2.5 mM Octane sulphonic acid). After sample injection buffer B was increased over a linear gradient of buffer A (milliQ water with 0.1% v/v H₃PO₄ and 2.5 mM Octane sulphonic acid) to 100% over 25 minutes. The retention time of synthetic ISF401 was 16.29 minutes and a standard curve using known amounts of ISF401 was established for quantification of unknown amounts of peptide in the urine samples.

Photo-diode array spectra and Matrix Assisted Laser Desorption Time Of Fight Mass Spectrometry (MALDI-TOF) were used to verify the molecular weight and peptide composition of the putative ISF401 in samples.

ISF401 conjugated to diphtheria toxoid via an amino terminal cysteine residue was used to immunize rabbits (Institute of Medical and Veterinary Science, Adelaide, Australia). Serum was collected after primary inoculation and 3 booster injections.

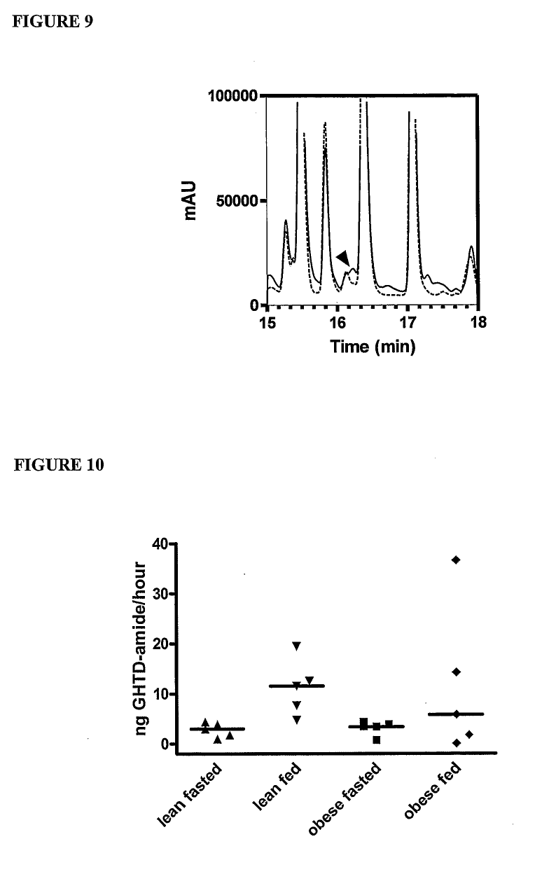

Detection of antigenic structures reactive with the ISF401 antiserum in pancreatic islet cells suggested an islet beta cell origin for urinary ISF401. This was tested using the mouse beta cell line MIN6. Serum free media was conditioned by confluent MIN6 cells for 24 hours. A species that absorbed at 214 nm with a retention time on C18 reverse phase HPLC (16.2 minutes) identical to that of synthetic ISF401 was identified in conditioned media but absent from unconditioned media (FIG. 9). MALDI-TOF mass spectrometry on the fraction collected over 16 to 17 minutes revealed a molecular weight of 428.983 Dalton and fragmentation produced the expected fragment sizes (Table 3).

TABLE 3
MALDI-TOF fragmentation of urinary peptide

| | Mass (Da) | | | | | | | |
|---|---|---|---|---|---|---|---|---|
| | 385 | 269.9 | 251.9 | 241.0 | 196.0 | 169.0 | 100.1 | 70.0 |
| Assignment | m-T1, | a3 | d3b | HT | b2 | a2 | d2a | w1a |
| | a4 | | | | | | | |

Thus ISF401 is secreted by MIN6 beta cells confirming pancreatic islet beta cells as an endogenous source of ISF401. The appearance of ISF401 in the media of MIN6 cells also indicates that the tetrapeptide is the form of the hormone secreted from beta cells rather than a breakdown product of a larger peptide. It would be anticipated that ISF401, like other peptide hormones, is processed from a larger precursor protein. Identifying this precursor through bioinformatic approaches is difficult due to the small size of the peptide and the high frequency of the GHTD sequence in the available databases (data not shown). Consequently, the biosynthetic pathway for ISF401 is currently not known.

Insulin is secreted in response to nutrient stimuli. Co-localisation of ISF401 with insulin would suggest that ISF401 will also be released in response to nutrients. This was tested by comparing the amounts of ISF401 excreted in urine of fasted and fed rats (FIG. 10). The experiment was performed in obese (fa/fa) Zucker rats, a model of insulin resistance and Type 2 diabetes, and their lean littermates in order to determine if ISF401 production was altered in a hyperinsulinemic animal model of insulin resistance and Type 2 diabetes. In lean Zucker rats the rate of ISF401 secretion increased 3-fold, whereas in obese Zucker rats two rats showed a large increase in urinary concentrations of the peptide while in 3 rats no increase in urinary ISF401 levels was apparent, with one rat lacking detectable peptide in the urine altogether (FIG. 10). Thus urinary excretion of ISF401 increases after feeding and in insulin resistant rats the amount excreted is highly variable.

### Example 9

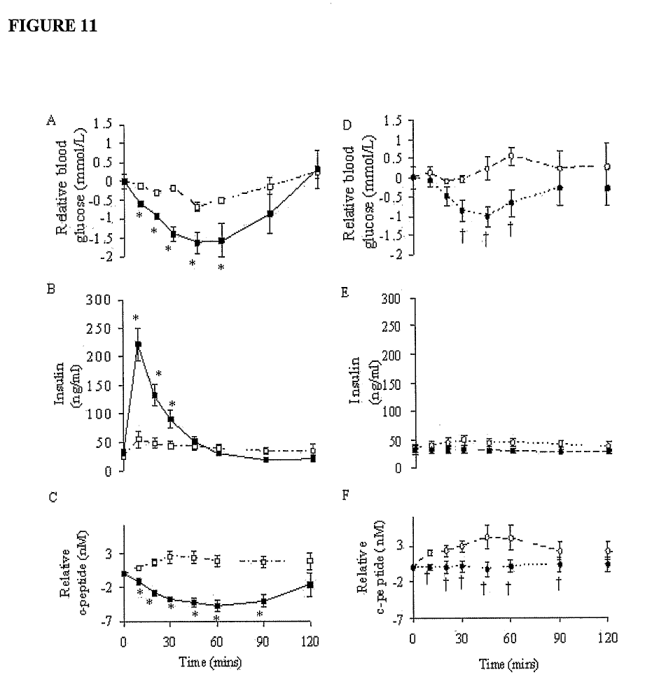

Examination of the time-course data (FIG. 11) for injected insulin and ISF402 at the optimum ISF402 dose of 1.5 mg/kg reveals the maximum reduction in blood glucose was 1.63±0.51 mmol/L at 45 minutes after administration compared to 0.68±0.22 mmol/L for insulin alone at the same time point (FIG. 11A). The increase in circulating insulin reached a peak 10 minutes after administration and remained elevated above insulin injected controls for 20 minutes (FIG. 11B). Calculation of the rate of disappearance of insulin from the circulation revealed a half-life of 13 minutes (9.9-13 minutes, 95 percent confidence interval). Simultaneously pancreatic insulin secretion as measured by serum C-peptide concentrations was decreased (FIG. 11C) and remained so for 90 minutes after injection of the peptide. Insulin alone had no effect on triglyceride levels whereas 10 minutes after injection of 1.5 mg/kg ISF402 with insulin there was a significant decrease in serum triglyceride compared to controls at the same time point (p<0.05, Table 4).

TABLE 4
Change in serum triglyceride levels compared to the 0 time point
in female Zucker fa/fa rats treated with ISF402 and/or Insulin

| | Change in Serum Triglyceride levels (mg/dl) | | | |
|---|---|---|---|---|
| | Time (minutes) | | | |
| | 0 | 10 | 45 | 60 |
| Saline | 0 | +24.1 ± 10.5 | +8.4 ± 4.0 | +22.1 ± 23.3 |
| ISF402 at | 0 | +11.3 ± 5.4 | −16 ± 7.2^{a} | +44.9 ± 42.7 |
| (4.5 mg/kg) | | | | |
| Insulin | 0 | +39.6 ± 15.1 | +21.1 ± 23.9 | +33.3 ± 19.8 |
| Insulin + | 0 | −15.0 ± 16.9^{b} | −3.4 ± 21.8 | +1.8 ± 21.0 |
| ISF402 | | | | |
| (1.5 mg/kg) | | | | |

Values are expressed as mean ± SEM (n = 5).
^{a}p = 0.03 ISF402 at 4.5 mg/kg compared to saline treated rats.
^{b}p = 0.04 Insulin and ISF402 at 1.5 mg/kg compared to insulin alone treated rats.

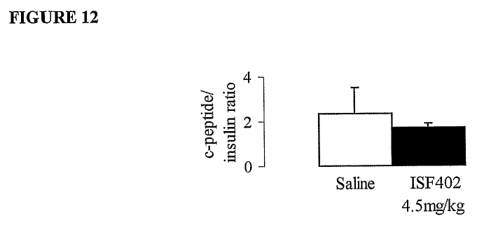

The optimal intravenous dose of ISF402 without the addition of exogenous insulin was 4.5 mg/kg. The time-course shows a maximum reduction in blood glucose of 1.02±0.27 mmol/L at 45 minutes after administration whereas there was no change in the saline injected controls (0.18±0.40 mmol/L) (FIG. 11D). The magnitude of the maximal reduction in blood glucose after injection of 4.5 mg/kg ISF402 was similar to that seen for co-injection of 1.5 mg/kg ISF402 with insulin when compared to insulin alone controls (0.95 and 0.94 mmol/L respectively). Injection of ISF402 at 4.5 mg/kg also reduced triglyceride levels 45 minutes after injection (P<0.05) (Table 4). The small decrease in serum insulin (FIG. 11E) and C-peptide concentrations (FIG. 11F) observed 10-45 minutes after administration did not translate into a difference in the rate of hepatic insulin clearance compared to the saline treated controls (FIG. 12).

### Example 10

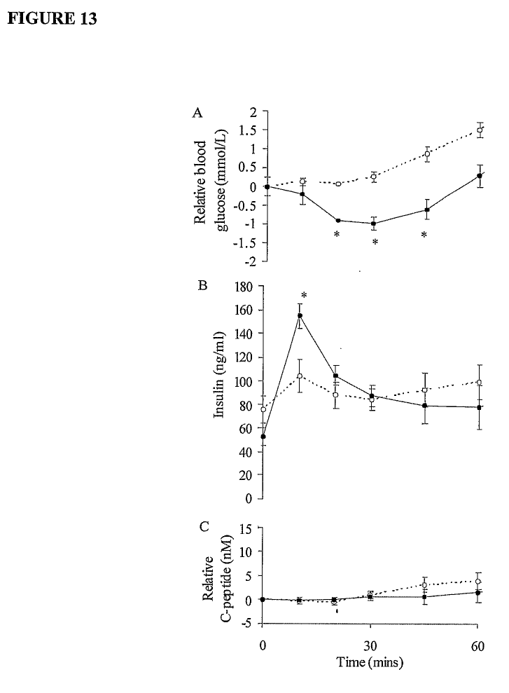

To investigate sex differences in the insulin sensitizing effect of ISF402, age-matched male Zucker fa/fa rats were injected intravenously with ISF402 (1.5 mg/kg) and insulin as described above for female rats. The male Zucker fa/fa rats showed a steady increase in blood glucose after injection of insulin alone, which is symptomatic of the profound degree of insulin resistance in these male rats. In comparison, co-injection of ISF402 with the insulin led to a decrease in blood glucose 20 minutes after injection which remained low for a further 25 minutes (FIG. 13A). The greatest reduction in blood glucose was 0.98±0.31 mmol/L and was achieved 30 minutes after injection. Male Zucker fa/fa rats commenced with an elevated basal insulin level compared to female Zucker rats and a peak of serum insulin was again apparent 10 minutes after injection of ISF402 with the insulin. However the magnitude was 30 percent lower in the males compared to the females (FIG. 13B). Serum C-peptide concentrations remained constant in both the control and ISF402 injected male rats (FIG. 13C), which is in contrast to the prolonged decline in C-peptide levels seen in females injected with ISF402. Thus in males Zucker fa/fa rats intravenous injection of insulin with ISF402 at 1.5 mg/kg effectively reduced blood glucose but had a lesser effect on serum insulin and C-peptide concentrations than was observed in females.

Male Zucker fa/fa rats displayed greater insulin resistance than females. Insulin resistance was apparent in both sexes by the lack of response to 1 U/kg of bovine insulin, which caused a small decrease in blood glucose in females and no decrease at all in males. Basal insulin concentrations were also high with serum insulin of 27±3 ng/mL in females and 75.8±11.3 ng/mL in males. By comparison the basal insulin level in lean Zucker rats is 1.67±0.42 ng/mL (Qu et al., J. Endocrinol., 162:207-214 (1999)). This suggests that the male Zucker fa/fa rats have a greater degree of insulin resistance than females. The body weight of male Zucker fa/fa rats is also higher than females and males usually develop diabetes more rapidly. This may be explained by the tendency of male Zucker fa/fa rats to accumulate visceral fat. Visceral fat is less sensitive to antilipolytic and re-esterification effects of insulin compared to subcutaneous fat (Kahn and Flier, J. Clin. Invest., 106:472-481 (2000)) and blood from visceral fat depots drains directly into the portal vein leading to increased free fatty acid flux to the liver (Hikita et al., Biochem. Biophys. Res. Commun., 277:423-429 (2000)) and impaired liver glucose metabolism, glucose intolerance, insulin resistance, insulin secretion and dyslipidaemia. In this context it is noteworthy that injection of ISF402 with insulin caused similar reductions in blood glucose in both male and female Zucker fa/fa rats, with both sexes displaying a 1 mmol/L decrease in blood glucose 45 minutes after administration compared to injection of insulin alone (FIGS. 2A and 13A) and increased circulating insulin 10 minutes after injection (FIGS. 2B and 13B). Unlike females however, serum C-peptide was unaltered in males, which may be attributable to the greater degree of insulin resistance and beta-cell dysfunction. These results show that ISF402 can overcome both moderate and severe insulin resistance as seen in female and male Zucker fa/fa rats respectively.

### Example 11

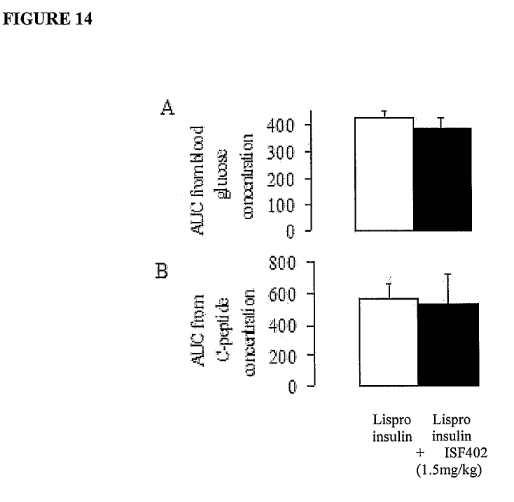

To test whether insulin sensitization and reduced serum C-peptide after co-injection of ISF402 with bovine insulin was related to dispersal of insulin hexamers by ISF402, an altered form of human insulin that does not form stable hexamers was used. Lispro insulin was injected with ISF402 at 1.5 mg/kg into 16-18 week old female Zucker fa/fa rats. Lispro insulin alone reduced blood glucose by 0.90±0.77 mmol/L 60 minutes after injection into female Zucker fa/fa rats. There was a slight reduction in blood glucose compared to controls as assessed by AUC when Lispro insulin was co-injected with ISF402 but this did not reach significance (FIG. 14A). Lispro insulin in serum could not be measured due to a high degree of variability between animals (not shown). Neither group showed a decrease in endogenous insulin release as shown by serum C-peptide concentrations (FIG. 14B). Thus, the insulin sensitizing activity of ISF402 was reduced when ISF402 was co-injected with Lispro insulin.

Lispro insulin differs from native human insulin in that the position of Pro and Lys at position B28 and B29, respectively are reversed hindering the formation of dimers—an intermediate step in hexamer formation. The monomeric property of Lispro insulin in in vivo enables its use in the treatment of diabetes as a fast acting insulin analogue. Co-injection of Lispro insulin and 1.5 mg/kg of ISF402 did not reduce blood glucose concentrations any further than injection of Lispro insulin alone (FIG. 14A) and C-peptide concentrations were also similar (FIG. 14B). Thus ISF402 may interact with injected hexameric insulin to promote the formation of monomeric insulin so making injected insulin immediately effective. When Lispro insulin is used in place of hexameric bovine insulin this effect is not observed as Lispro insulin is already in monomeric form. Notably, injection of 1 U/kg body weight of Lispro insulin alone caused a greater reduction in blood glucose and C-peptide concentration than did 1 U/kg body weight of bovine insulin, consistent with the notion that monomeric insulin is more effective than hexameric insulin at reducing blood glucose after intravenous injection. However, this cannot explain the reduction in blood glucose after injection of ISF402 alone, suggesting that ISF402 also has insulin-independent effects that promote insulin sensitivity.

### Example 12

### Solubility of ISF402

Solid ISF402 was added to 50 μL aliquots of 25 mM ammonium bicarbonate buffer until no more solid dissolved. The total weight of the peptide added to each tube was noted and the pH of each solution was adjusted to approximately pH 8 by the addition of 1 M NaOH. Samples were incubated with constant shaking at either room temperature (23-25° C.) or 37° C. for 24 hours then centrifuged at 16000×g for 15 minutes. 2 μL aliquots of each supernatant were diluted with milliQ-H₂O to an estimated concentration of 1 mg/mL. Samples were stored at −20° C. for later analysis by HPLC using UV detection (214 nm) to determine the exact concentration of ISF402. The pH of the remaining supernatant was measured. The pH was lowered by the addition of 2 μL of 5M HCl and tube contents were mixed and incubated under the two temperature conditions. The cycle of pH lowering, incubation and sampling was repeated until a pH of between 2 and 3 was achieved.

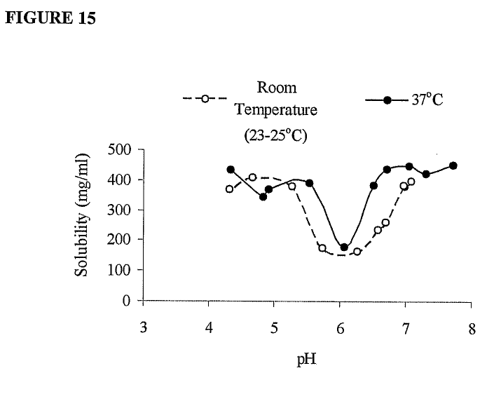

The solubility of ISF402 both at room temperature and 37° C. was between 300-450 mg/mL below pH 4.6 and above pH 6.9. Solubility at both temperatures decreased between pH 4.6-6.9 with a minimum solubility observed at pH 5.7-6.3, which is close to the theoretical isoelectric point of the peptide (pH 6.71) [ExPASy. Compute pI/Mw tool. Available at http://ca.expasy.org/tools/pi_tool.html, accessed Mar. 31, 2006]. The lowest solubility recorded for ISF402 at room temperature was 165 mg/ml at pH 6.3 while the minimum solubility at 37° C. was 179 mg/mL at pH 6 (FIG. 15).

### Example 13

### Stability of ISF402

Simulated gastric fluid (SGF) and simulated intestinal fluid (SIF) were formulated according to the British Pharmacopeia (British Pharmacopoeia, 1988, Her Majesty's Stationary Office, London). SGF contained 0.1 M NaCl, 32 mg Pepsin (Sigma, St. Louis, Mo.) in 50 mL of distilled water (dH₂O) with 4 mL of 2M HCl, pH at 1-1.3 and adjusted to 100 mL of dH₂O, SIF contained 156 mM KH₂PO₄, 18.6 mM NaOH, 1 g/L Pancreatin (Sigma, St. Louis, Mo.) pH 6.8 and adjusted to 10.0 mL with dH₂O. Each fluid was equilibrated to room temperature before the addition of bovine serum albumin (BSA) or 1 mg/mL ISF402. The mixtures were mixed gently and samples were taken at the indicated time points and stored at −80° C. until analysed.

BSA samples were analysed by electrophoresis on a 10 percent polyacrylamide non-reducing SDS-gel and stained with Coomassie Blue. ISF402 samples were analysed by reverse phase HPLC using a Waters system coupled with a Waters 440 absorbance detector with extended wave length module at 214 nm. The analytic column was a Phenomenex luna (2)-c-18 column, 250×46 m, 5 μm particle size. The solvents were degassed and the buffers used were Buffer A: 100 percent milliQH₂O, 0.1 percent v/v H₃PO₄, 2.5 mM Octane sulphonate and Buffer B: 90 percent Acetonitrile(_{aq}), 0.1 percent v/v H₃PO₄, 2.5 mM Octane sulphonate. 50 μl of ISF402 in SIF and SGF were injected and eluted with a linear gradient at 10 percent B for 2 minutes and 10-100 percent of B over 25 minutes. The column was cleaned between runs with 100 percent B for 13 minutes and equilibrated with 10 percent B for 10 minutes. The retention time (in minutes) and the area under the peak of ISF402 were plotted against reaction time course.

Mass spectroscopy for ISF402 in SGF and SIF reaction mixtures were determined by direct injection of ISF402 peak fractions into the Electrospray spectrometer in the positive ion mode using a cone voltage of 30V. Electrospray ionization mass spectroscopy (ESI-MS) was performed on the 420 minute SGF and SIF samples. The positive ion of ISF402 has a molecular mass of 470.0 Daltons.

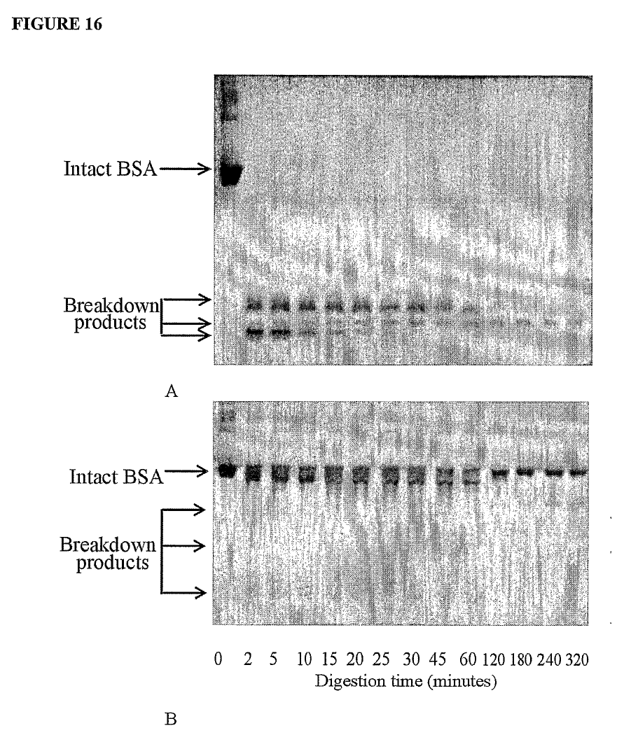

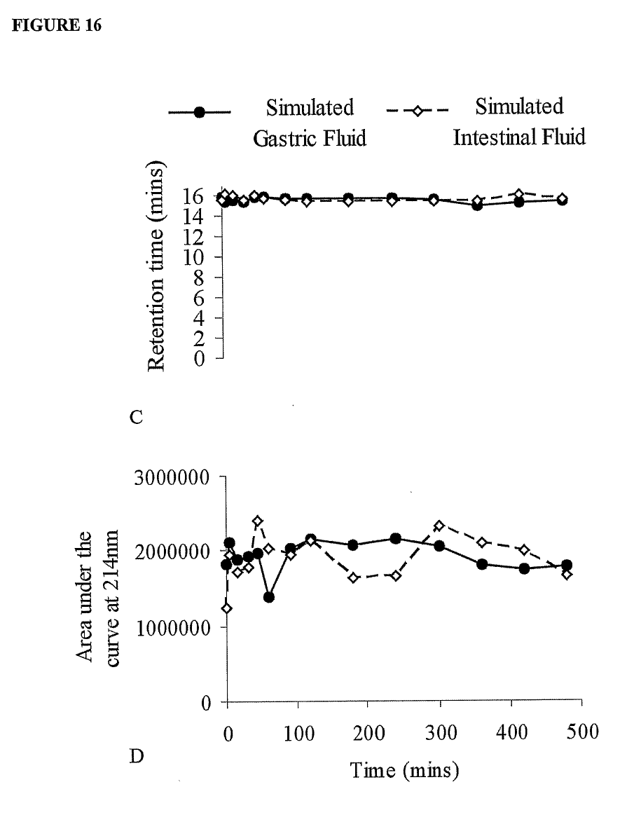

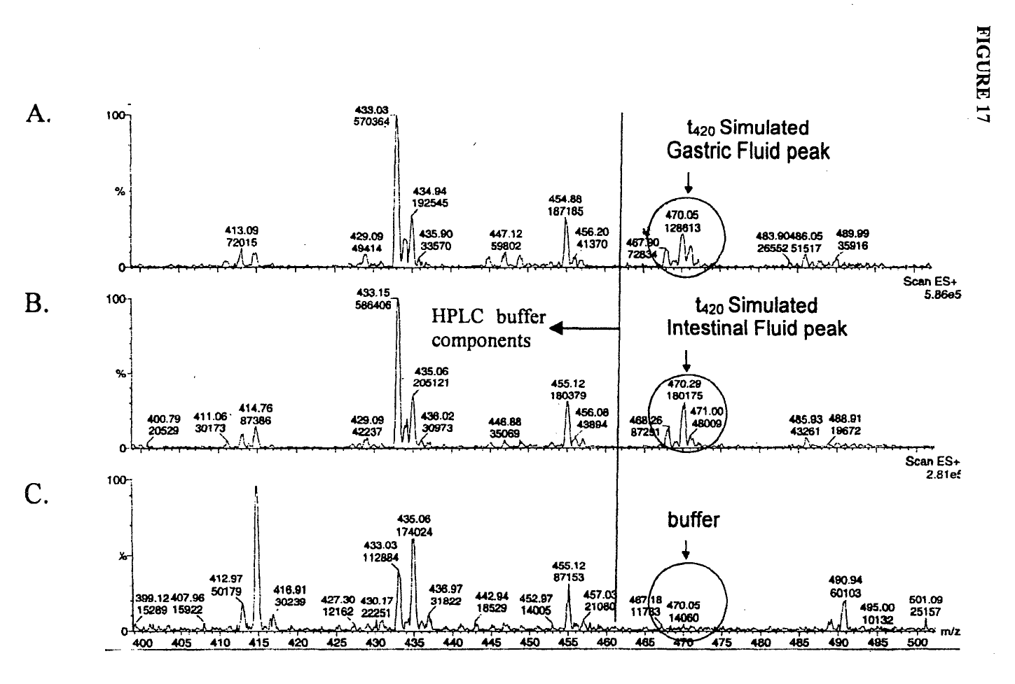

BSA was degraded within 2 minutes in SGF (FIG. 16A). The activity of pancreatin in the SIF was shown by the appearance of a smear below the BSA band and the appearance of lower molecular degradation products after longer incubations (FIG. 16B). ISF402 did not degrade after 8 hours incubation in SGF and SIF as determined by reverse phase HPLC retention time and peak size (FIGS. 16C and D). The chemical integrity was confirmed by ESI-MS. After 7 hours incubation in SGF or SIF the molecular mass of ISF402 was unchanged (FIGS. 17A and B). ESI-MS of HPLC buffer blanks showed that constituents with a lower molecular mass than ISF402 were attributable to buffer components rather than fragments of ISF402 (FIG. 17C).

### Example 14

### Insulin Sensitisation by ISF402

Female Zucker fa/fa rats were purchased from Monash Animal Services (Monash University, Clayton, Australia). Rats were housed in the Biochemistry and Molecular Biology Animal House (Monash University, Victoria, Australia) and allowed to acclimatize for 7 days in an environmentally controlled room at 22° C. Rats were fed normal chow and water ad libitum. All experiments were performed according to Monash University Animal Care and Ethics Committee guidelines and approved by the Monash University Animal ethics committee.

Rats of 16-18 weeks of age were fasted overnight with free access to water then anaesthetised with pentobarbitone (Nembutal, Phone Merieux, QLD, Australia) administered intraperitoneally (IP) at 35 mg/kg body weight. Blood glucose was monitored using Medisense glucometers (Abbott Laboratories, Abbott Park, Ill.) for one hour prior to administration of test substances. ISF402 was administered at 3 mg/kg by IP with 2 U/kg of bovine insulin (n=6) and controls were injected IP with insulin alone (n=8). Two experiments were performed where ISF402 was given orally. In the first experiment, controls were given saline orally by gavage (n=5) and the test group were given ISF402 orally at 15 mg/kg (n=5). After 15 minutes, insulin (2 U/kg) was injected IP. The procedure was repeated three days later with the control rats once again given saline and the treated rats given 30 mg/kg ISF402. A second experiment was performed on a separate group of rats where controls were given saline orally as before (n=5) and treated rats were given ISF402 orally at 30 mg/kg (n=5), followed 15 minutes later by IP injection of 2 U/kg insulin. For both experiments, after administration of peptide blood was collected from the tail vein and glucose measured immediately. Serum samples were also collected for later measurement of C-peptide and insulin using Linco Rat C-peptide and Insulin RIA kits according to the manufacturer's instructions (Linco Research Inc, St Charles, Mo.).

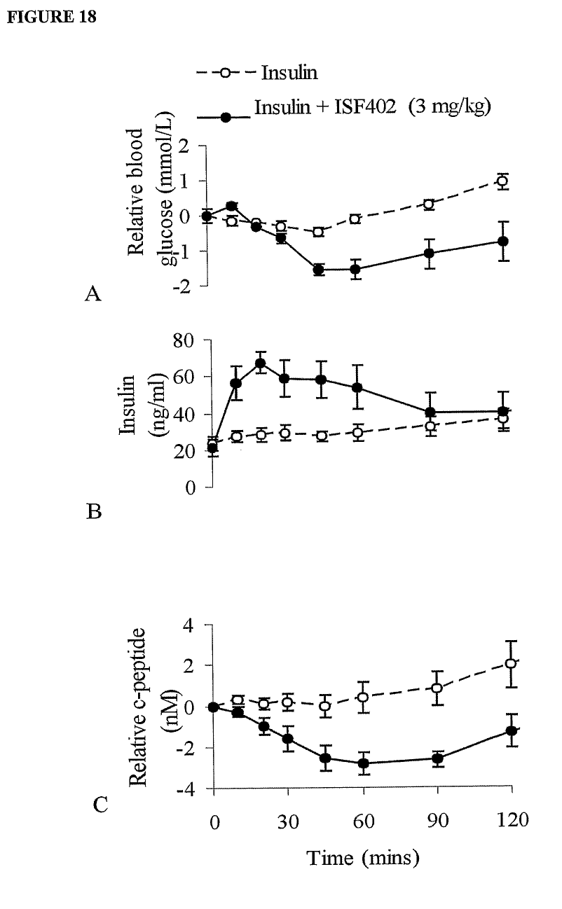

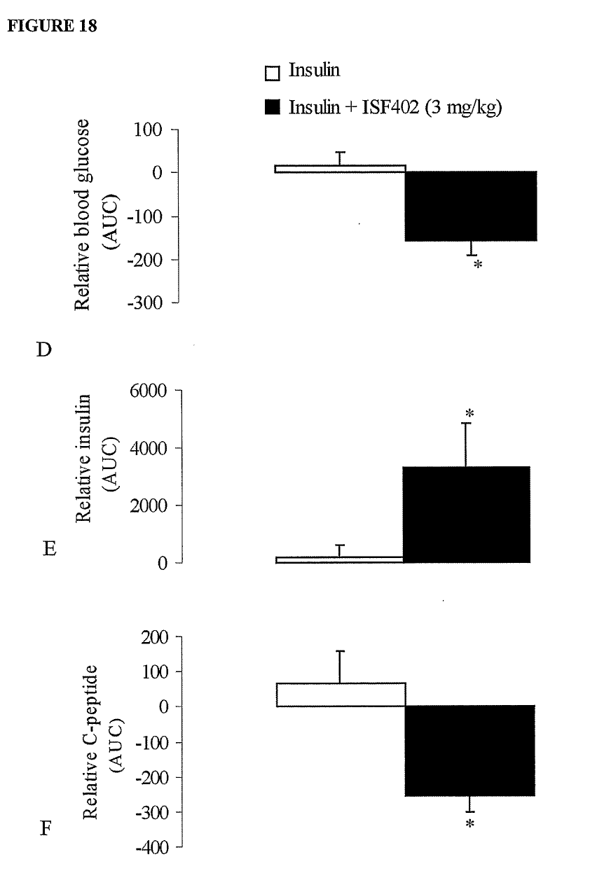

The Zucker fa/fa rat was selected to test insulin sensitizing activity of ISF402 due to its similarities to human Type 2 Diabetes including insulin resistance and hyperinsulinemia. IP injection of 2 U/kg bovine insulin caused only a small reduction in blood glucose demonstrating the extreme insulin resistance characteristic of Zucker fa/fa rats (FIG. 18A). However IP injection of 3.0 mg/kg of ISF402 with the insulin led to a significant reduction of blood glucose (FIG. 18A). The maximum reduction in blood glucose was 1.56±0.42 mmol/L at 45 minutes after injection and lower blood glucose readings were sustained for more than an hour. Furthermore, 20 minutes after injection of ISF402 circulating insulin concentrations were 2 fold higher compared to injection of insulin alone and remained elevated for 40 minutes (FIG. 18B). Simultaneously pancreatic insulin secretion was decreased from 20-60 minutes after injection of the peptide as measured by serum C-peptide concentrations (FIG. 18C).

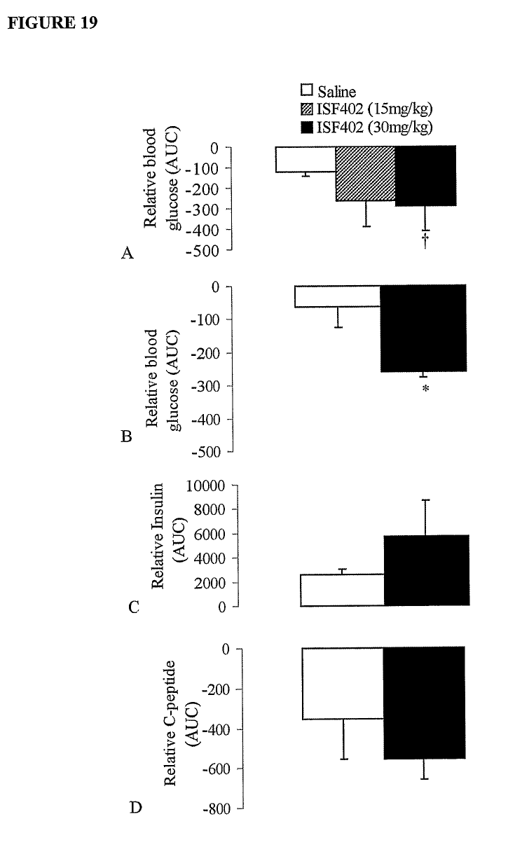

A dose of 15 mg/kg of ISF402 was orally administered and 15 minutes later an IP insulin tolerance test (IPITT) was performed. Fourteen (14) minutes after IPITT (29 minutes after administration of ISF402) there was a trend towards decreasing blood glucose but the results were variable and did not reach statistical significance (FIG. 19A). Three days later a dose of 30 mg/kg of ISF402 was administered orally to the same rats and the IPITT repeated, which produced a significant reduction in blood glucose compared to the insulin-injected control group (FIG. 19A). To investigate whether the repeated dose contributed to the increased insulin sensitivity of the second dose, the experiment was repeated on a different group of rats. In this experiment a single dose of 30 mg/kg of ISF402 was given orally by gavage and an IPITT performed as before. There was a significant decrease in blood glucose that persisted for the duration of the time course (FIG. 19B). Unlike IP injection of ISF402 and insulin, the reduction in blood glucose after oral ISF402 was not associated with increased circulating insulin or reduced C-peptide concentrations (FIGS. 19C and D, respectively).

### Example 15

### Administration of ¹⁴C-ISF402 n Female Obese Zucker fa/fa Rats

¹⁴C-ISF402 (22.7 μCi/mg) was dissolved in water and diluted with unlabelled ISF402 to a concentration of 30 mg/mL for oral and 4.5 mg/mL for intravenous (IV) administration. The day before the experiment 16-18 week female Zucker fa/fa rats (300-400 g) rats were fasted overnight with free access to water before oral administration of ¹⁴C-ISF402 by gavage at 30 mg/kg, 260-263.7 kBq/kg (5.0-5.1 μCi/kg) or IV with 4.5 mg/kg, 103.0-107.3 kBg/kg (1.98-2.06 μCi/kg). After administration of ¹⁴C-ISF402 rats were placed into metabolic cages with free access to food and water.

Blood samples were collected from the tail vein over the 7 hour time course. Urine was collected at 4 and 12 hours after administration. Whole blood samples (100 μL) were collected and placed into dry ice then stored at −80° C. 50 μL of serum from whole blood was collected into T-MG clotting capiject tubes (Terumo, Elkton, Md.). Sera were separated by centrifuging blood for 90 seconds at 3000×g at room temperature. 100 μL and 50 μL of whole blood and serum, respectively was used for the measurement of radioactivity. The whole blood and serum samples were solubilized with 2 volumes of sample, 2 volumes of 1.4M NaOH and 1 volume of 30 percent hydrogen peroxide. The final volume was made to 1 mL by the addition of distilled water. 4 mL of HiSafe3 Optiphase (PerkinElmer, Boston, Mass.) was added and radioactivity determined in a Wallac Scintillation counter.

Urine was frozen at −20° C. until analysed for radioactivity. After measuring the total volume of urine, 100 μL of urine and 100 μL ethanol was added to 4 mL scintillant. Samples were left overnight after the addition of scintillant then counted for radioactivity using a Wallac 1409 liquid scintillant counter (PerkinElmer, Boston, Mass.). Radioactivity in all samples was counted several times over the course of 4 weeks until readings were consistent. Radioactivity was expressed as μg equivalent (eq.)/mL. This was calculated from the dpm per mL of sample divided by the specific radioactivity of the peptide administered. The integrity of the radiolabelled peptide in serum and urine samples was determined by reverse phase HPLC as described above.

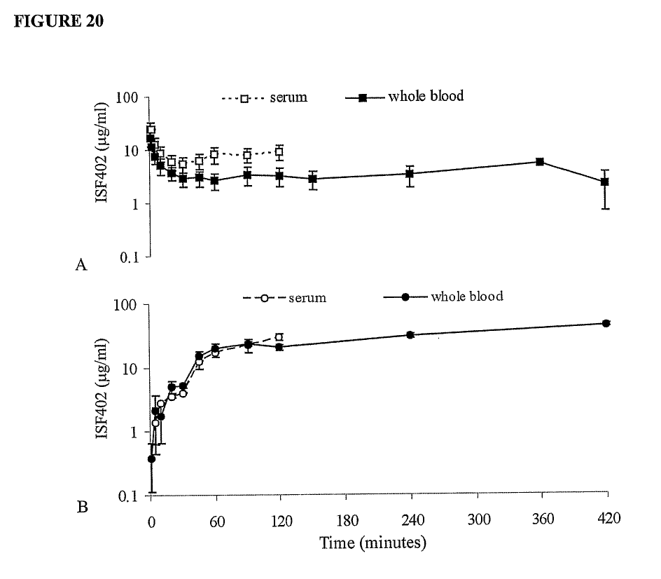

Two (2) minutes after IV injection of ¹⁴C-ISF402 in the femoral vein of the Zucker fa/fa rats ISF402 was detected in whole blood at a concentration of 12±1 μg/mL and most of this reactivity was in the serum (concentration in serum 24.8±0.3 μg/mL) (FIG. 20A). Ninety (90) minutes after injection radioactivity in the circulation decreased to 3.4±1.3 μg/mL in whole blood (8±2 μg/mL in serum), assuming the radioactivity is attributable to intact ISF402 and at the conclusion of the time course (i.e. 7 hours) 2.2±1.5 μg/mL of ISF402 was detected. After oral administration, ¹⁴C-ISF402 appeared in the circulation within 30 minutes after dosing and radioactivity in the circulation gradually increased with time (FIG. 20B). After 90 minutes levels corresponding to 23.7±2.1 μg/mL, assuming the labelled ISF402 was still intact, were detected in whole blood and similar concentrations (22.1±4.9 μg/mL) were detected in sera. At the conclusion of the ¹⁴C-ISF402 in blood-time course (7 hours) 44.2±2.5 μg/mL of ISF402 was detectable in whole blood.

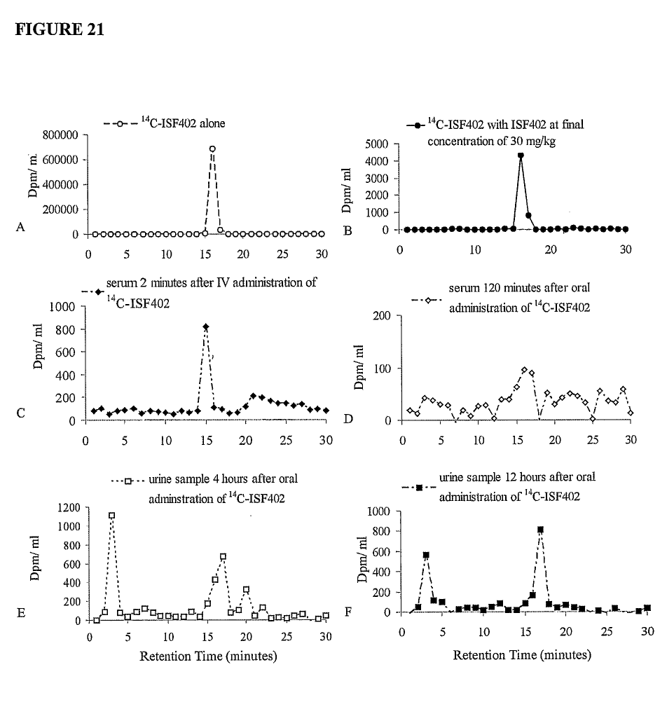

To determine whether the radioactivity corresponds to intact or degraded peptide, urine and serum samples were separated by reverse phase HPLC and the profiles compared to intact ¹⁴C-ISF402. Intact ¹⁴C-ISF402 (FIG. 21A) and the mixture of labelled and unlabelled ISF402 administered to the rats (FIG. 21B) both elute in fractions 15 to 17. The elution profiles in serum collected 2 minutes after IV administration identifies a peak at 14-17 minutes indicating that the ISF402 is intact (FIG. 21C). Elution profiles of the radioactivity in a serum sample collected 120 minutes after oral administration shows a small peak at 15 to 17 minutes corresponding to approximately 27 percent of the total radioactivity in the sample (FIG. 21D), although the amount of radioactivity present was close to the limits of detection. Urine collected over 4 hours after dosing contained a mixture of degraded peptide and intact ISF402 with the majority of the radioactivity eluting in fractions 3, corresponding to free Valine, and 15-17 which represents intact ISF402 (FIG. 21E). A third peak was also present at a retention time of 20 minutes, which may represent another degradation product or ISF402 bound to another molecule.

Interestingly after 12 hours of administration there were only two peaks apparent. Most of the radioactivity (47 percent) was associated with intact ISF402 while 32 percent was associated with free Valine (FIG. 21F). At the conclusion of the ¹⁴C-ISF402 urine-time course (i.e. 12 hours) the average ¹⁴C-ISF402 retrieved in urine was 0.97±0.13 percent of the total radioactive ISF402 administered.

### Discussion for Examples 12-15

Many peptide drug candidates are not effective when given orally due to digestion by intestinal peptidases and poor permeability across the intestinal epithelium. Here it is shown that the insulin sensitizing tetrapeptide ISF402 resists proteolytic degradation and effectively improves insulin sensitivity in rats when administered orally.

A high degree of solubility and stability are important if an orally administered drug is to be absorbed across the intestinal wall and enter the portal vein intact. The driving force for diffusion across the apical and basolateral membranes of the enterocyte is dependent on the solubility of the drug and the concentration gradient, and for ionizable drugs this varies with the pKa and the pH profile between the intestinal compartments. ISF402 was highly soluble in aqueous solution and solubility varied with pH in a manner typical of zwitterionic peptides and drugs (Pasini and Indelicato, Pharm. Res., 1992, 9:250-254). Drug stability is of equal importance. Proteolysis in the stomach often destroys the peptide before it reaches the intestine for absorption. Polypeptides are usually degraded to protein fragments and free amino acids by the action of gastric and pancreatic enzymes. ISF402 is an exception and was able to withstand prolonged incubation in simulated gastric and intestinal fluids. However, ISF402 may still be susceptible to intestinal and brush border peptidases, which must be encountered before entry into the hepatic portal vein.

A major limitation of oral administration is the lack of retention of the dosage form at the site of absorption due to continuous dilution by digestive fluids (Weatherell et al., Oral Mucosal Drug Delivery, NY, Marcel Dekker, 1996, p 157-191). Taking into consideration this dilution effect and the possibility of degradation by brush border peptidases upon passage across the intestine the oral dose initially chosen to test oral efficacy of ISF402 was 5 to 10 times higher compared to the intraperitoneal route. A dose of 15 mg/kg showed a trend towards increased insulin sensitivity but this did not reach significance. When the oral dose was increased to 30 mg/kg and administered to the same rats 3 days later there was a significant increase in insulin sensitivity as assessed by IPITT. There appeared to be no cumulative effect of the 2 doses of ISF402, since a second experiment administering 30 mg/kg orally to a another group of Zucker fa/fa rats produced similar reductions in the glucose profile as in the repeat dose experiment. It is noteworthy that IP injection of ISF402 with insulin resulted in increased circulating insulin and reduced C-peptide, similar to our previous report on IV injection. Oral ISF402 followed by IP insulin injection however did not change circulating insulin and C-peptide concentrations. These results suggest a direct interaction between injected insulin and ISF402 that does not occur when the two are administered by separate routes. Nevertheless, oral delivery of ISF402 was still effective in improving insulin sensitivity as assessed by IP insulin tolerance testing.

Within 30 minutes after oral administration of ¹⁴C-ISF402 radioactivity could be detected in the circulation. Evidence that between 25 and 50 percent of this radioactivity was still associated with intact ISF402 peptide comes from the RP-HPLC elution profiles. Approximately 4 μg/mL of ISF402 was detected in whole blood and sera 30 minutes after oral dosing and after 120 minutes ISF402 levels increased to more than 20 μg/mL, at which time approximately 25 percent of radioactivity represented intact ¹⁴C-ISF402. Further evidence of the stability of ISF402 comes from the observation that 46 percent of the radioactivity retrieved in urine collected over 12 hours after administration was intact ISF402. These data indicate that the amino terminal valine is cleaved from 50 to 75% of the ISF402 during passage into the circulation. This limits the interpretation of the concentration-time profiles, particularly since the radiolabelled valine may be incorporated into newly synthesised proteins and re-enter the circulation. However, the data also show that between 25 and 50% of the absorbed ISF402 was able to withstand the various gastrointestinal tract environments and passage across the intestine to enter the circulation intact (FIG. 21F). However, IPITT suggested the greatest decrease in blood glucose after oral administration of 30 mg/kg ISF402 occurred 2 hours after administration, which corresponds to the time of maximal concentration in the circulation.

### Example 16

### ELISA Detection of ISF401 in Urine

In an alternative methodology, ISF peptides may be detected in biological fluids using ELISA assay techniques.

Methods

As examples, the method as it applies to ISF401 and ISF402 is described. The method could reasonably apply to any ISF peptide described in this specification and a person skilled in the art would be able to adapt the assay described so that it is suitable for other ISF peptides.

Polyclonal antibodies were raised in New Zealand White rabbits by multiple subcutaneous injections of 0.5 mg of ISF401 or ISF402 conjugated to diphtheria toxoid conjugate (ISF-diptox). Conjugation to diphtheria toxin was by addition of an N-terminal cysteine to the peptides and a Maleimidocaproyl-N-Hydroxysuccinimide (MCS) linker. ISF-diptox was emulsified prior to injection with complete Freund's adjuvant (Institute of Medical and Veterinary Science, Adelaide, Australia). Antisera were collected after the eighth week once the third and final immunisation was completed.

Streptavidin coated plates were blocked with 2 percent casein in phosphate buffered saline (PBS) at pH 7.2 (0.1 M sodium phosphate 0.15 M sodium chloride, pH 7.2) (Casein blocking solution) (200 μL/well). Plates were incubated at 25° C. hour for 1 hour with constant mixing. Plates were washed four times in PBS containing 0.1 percent Tween-20 (PBST). Streptavidin-blocked plates were coated with 100 μL of 5 μg/mL biotinylated ISF401 or ISF402. Biotinylate peptides were biotin-SGSG-GHTD-NH₂ or biotin-SGSG-VHTD-NH₂. After incubation plates were washed with PBST as before.

Serial dilutions of ISF from 50 μg/mL to 6.1 pg/mL in casein blocking solution were used to generate a standard curve for the competitive inhibition assay. The peptide solutions and serum and urine test samples containing known quantities of ISF401 or ISF402 were pre-incubated at 80° C. for 15 minutes in eppendorf tubes and then mixed with an equal volume of 1/3200 dilution of antiserum and incubated at 25° C. for 45 minutes. The peptide-antibody mixture was added to wells of ISF-biotin/streptavidin coated plates and held at 25° C. for an hour with constant mixing. The wells were washed four times with PBST and then incubated with 100 μL/well of 1/4000 HRP conjugated anti-rabbit antibody at 25° C. for 1 hour. The plates were washed four times with PBST and once with PBS. Colour was developed by addition of 100 μL/well of substrate solution for 20±5 minutes at 25° C.

Results and Discussion

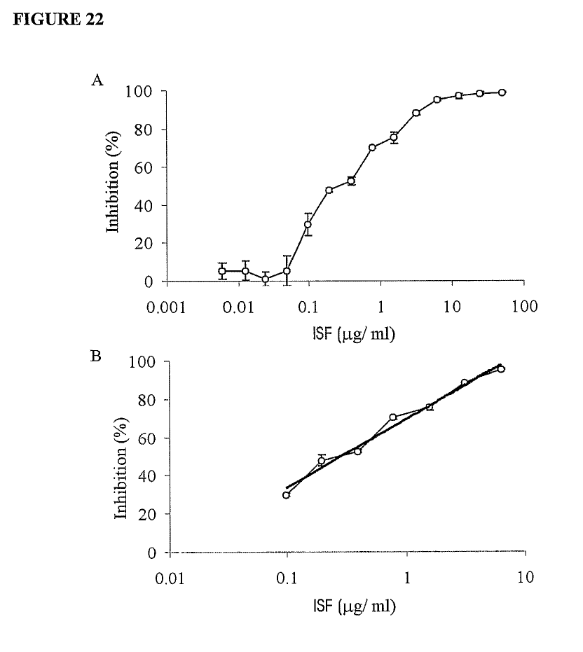

Inhibition of ISF antibody binding to immobilised biotin-ISF by free ISF peptide was apparent down to concentrations of 97.6 pg/mL (FIGS. 22A and B). At concentrations above 6.25 μg/mL inhibition was near to 100 percent. The linear regression coefficient for the linear portion of the sigmoidal inhibition curve (97.6 pg/mL to 6.25 μg/mL) was 0.98 (FIGS. 22A and B).

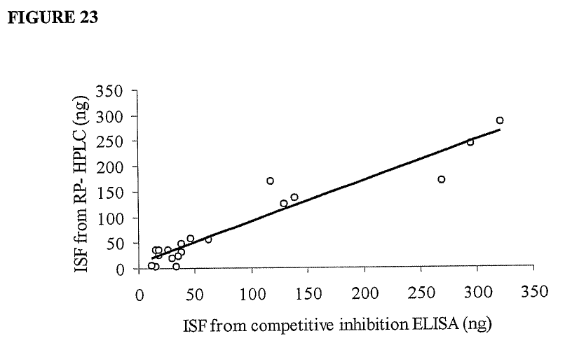

The utility of the CI-ELISA for measurement of urinary ISF was tested in various rat urine samples and the results compared to those using RP-HPLC and detection of eluted peptides at 214 nm using methods described in example 10. The urinary ISF was quantitated by comparison of the area under the curve of the 16.2 to 16.3 minute peak with a standard curve of known amounts of ISF. Comparison of these results with the CI-ELISA results for the same raw urine samples showed a significant correlation (r²=0.92) (FIG. 23).

### Example 17

### Methods

Size exclusion chromatography was used to study the effect of GHTD-amide (ISF401) on hexameric insulin. Recombinant Human Insulin solution (Sigma 19278) was diluted to 2 mg/mL with 10 mM Tris pH 7.4 and dialysed against 10 mM Tris pH 7.4 (to remove HEPES which absorbs strongly at 214 nm and coelutes with GHTD-amide). Insulin stock solution at 1.5 mg/mL was then prepared by addition of phenol to 4 mM, NaCl to 140 mM and ZnCl₂ to 100 μM. Prior to size exclusion chromatography stock insulin was mixed either with water or test peptide to give a final concentration 1 mg/mL each of insulin and test peptide when applicable, and 10 mM Tris pH 7.4, 140 mM NaCl and 100 mM ZnCl₂ then incubated at room temperature for 1 hour. Samples of insulin or mixtures (800 μL) were then subjected to size exclusion chromatography using a 1×30 cm Superdex 75 HR 10/30 column at a flow rate of 0.1 mL min⁻¹ with an eluent comprising Tris-buffered isotonic saline (140 mM NaCl, 10 mM Tris/HCl pH 7.4, 60 μM ZnCl₂) at a flow rate of 0.1 mL min⁻¹, UV detection at 214 nm and 276 nm, and collection of 0.5 mL fractions for protein determination. Protein size standards Aprotinin (6 kDa) and Carbonic Anhydrase (29 kDa) run under the same conditions eluted at 18.85 mL and 14.75 mL respectively.

The gel matrix, which has a fractionation range of 3,000 to 70,000 Da, and sample volume loaded were chosen to ensure that the two peptides remained in contact within the gel matrix thereby allowing interaction to occur between the two during the separation process. The concentration of insulin was maintained at 1 mg/mL at pH 7.4 in the presence of 2 Zn²⁺/hexamer since it has been shown that at this concentration insulin exists predominantly as Zn²⁺-dependent hexamers.

Results

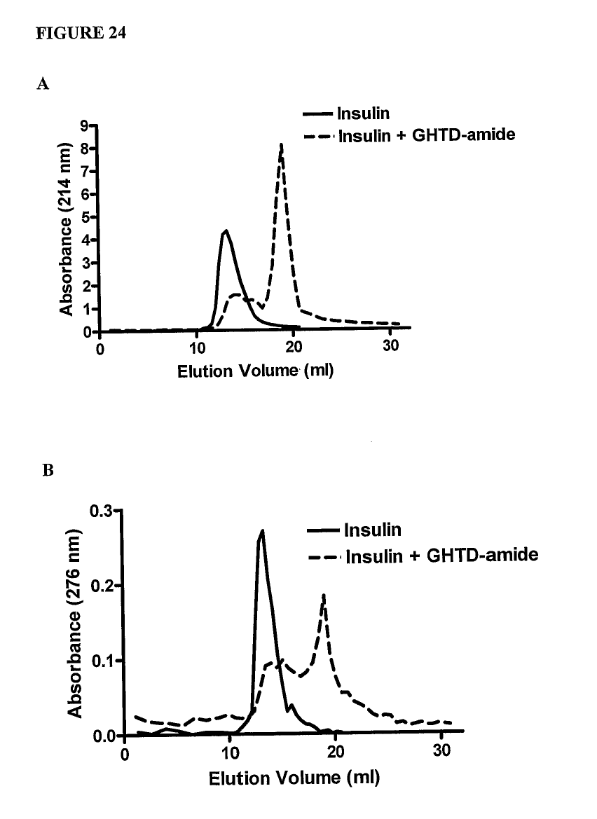

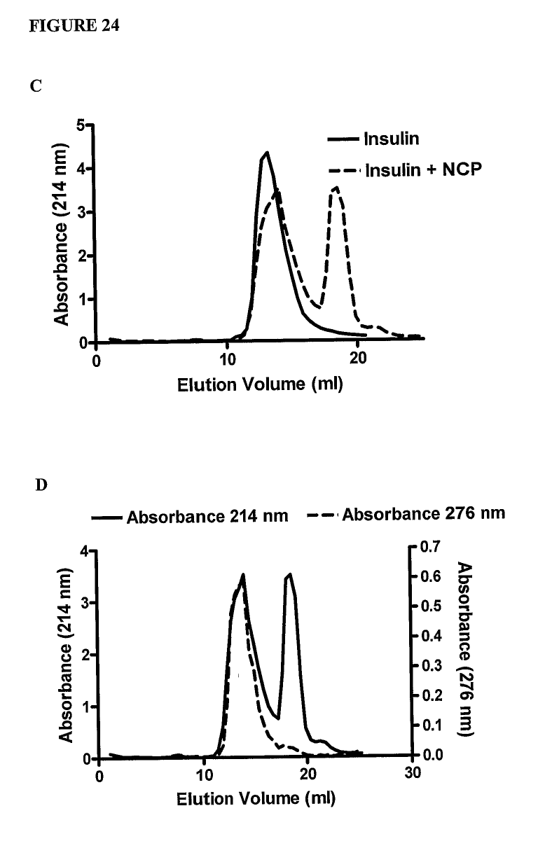

Size exclusion chromatography of human insulin alone results in the elution of hexameric insulin as a single peak at 13.315 mL (FIG. 24A). Following incubation of insulin with GHTD-amide, separation by size exclusion chromatography and monitoring of the eluant at 276 nm, the amount of hexameric insulin is reduced as shown by the reduction and broadening of the peak at 13.39 to 13.95 mL. The two minor peaks appearing at 15.085 mL and 19.045 mL correspond to dimeric insulin (11.8 kDa) and monomeric (5.8 kDa) insulin respectively (FIG. 24B).

The elution profile at 214 nm of a mixture of insulin and NCP a control tetrapeptide which does not chelate zinc showed the presence of two distinct peaks of similar size (FIG. 24C). The first which extends from 13.21 to 14.09 mL corresponds to hexameric insulin and the second at 18.56 mL corresponds to NCP. Monitoring of the elution of insulin and NCP at 276 nm (FIG. 24D) shows that the peak at 13.21 to 14.09 mL consists of hexameric insulin and there is no insulin present in the peak at 18.56 mL. These results were confirmed by protein analysis of fractions by the Bradford method which detects insulin but neither GHTD-amide nor NCP.

Hence the addition of the zinc-binding peptide GHTD-amide to hexameric insulin interacts with insulin to cause the dissociation (disaggregation) of hexamers to dimeric and monomeric forms. Addition of a peptide which did not chelate zinc did not cause dissociation of hexameric insulin.

1. A composition comprising a peptide of the formula:
(Xaa)ₙ₁-Xaa₁-His-Thr-Asp-(Xaa)ₙ₂
wherein
Xaa is any amino acid;
Xaa₁ is a hydrophobic amino acid;
n₁ is 0-10; and
n₂ is 0-10; and derivatives thereof;
and insulin.

2. A composition according to claim 1, wherein the peptide is one of the formulae:
(Xaa)ₙ₁-Val-His-Thr-Asp-(Xaa)ₙ₂; or
(Xaa)ₙ₁-Gly-His-Thr-Asp-(Xaa)ₙ₂;
wherein Xaa, n₁ and n₂ are as defined above and derivatives thereof.

3. A composition according to claim 1, wherein the peptide is Gly-His-Thr-Asp or a C-terminal and/or N-terminal capped derivative thereof.

4. A composition according to claim 1, wherein the insulin is derivatised, synthetic or recombinant human insulin.

5. An insulin-peptide complex in which the insulin is associated with at least one peptide of the formula:
(Xaa)ₙ₁-Xaa₁-His-Thr-Asp-(Xaa)ₙ₂
wherein
Xaa is any amino acid;
Xaa₁ is a hydrophobic amino acid;
n₁ is 0-10; and
n₂ is 0-10; and derivatives thereof.

6. An insulin-peptide complex according to claim 5, wherein the insulin:peptide ratio is 1:1 or 2:1.

7. A method of preparing a very fast acting insulin composition comprising the step of mixing a multimeric insulin complex with peptide of the formula:
(Xaa)ₙ₁-Xaa₁-His-Thr-Asp-(Xaa)ₙ₂
wherein
Xaa is any amino acid;
Xaa₁ is a hydrophobic amino acid;
n₁ is 0-10; and
n₂ is 0-10 and derivatives thereof.

8. A method of dispersing multimeric insulin complexes comprising the step of exposing multimeric insulin complexes to a peptide of the formula:
(Xaa)ₙ₁-Xaa₁-His-Thr-Asp-(Xaa)ₙ₂
wherein
Xaa is any amino acid;
Xaa₁ is a hydrophobic amino acid;
n₁ is 0-10; and
n₂ is 0-10 and derivatives thereof.

9. A method according to claim 8, wherein the multimeric insulin complexes are dimeric or hexameric insulin complexes.

10. A method according to claim 9, wherein the multimeric insulin complexes are hexameric insulin complexes.

11. A method of regulating in vivo blood glucose levels in a human or other mammal, which comprises administration of a combination comprising insulin and a peptide of the formula:
(Xaa)ₙ₁-Xaa₁-His-Thr-Asp-(Xaa)ₙ₂
wherein
Xaa is any amino acid;
Xaa₁ is a hydrophobic amino acid;
n₁ is 0-10; and
n₂ is 0-10 and derivatives thereof.

12. A method according to claim 11, wherein the peptide is one of the formulae:
(Xaa)ₙ₁-Val-His-Thr-Asp-(Xaa)ₙ₂; or
(Xaa)ₙ₁-Gly-His-Thr-Asp-(Xaa)ₙ₂;
wherein Xaa, n₁ and n₂ are as defined.

13. A method according to claim 12, wherein the peptide is a tetrapeptide selected from
Val-His-Thr-Asp (ISF402); and
Gly-His-Thr-Asp (ISF401);
or C-terminal and/or N-terminal capped derivatives thereof.

14. A method according to claim 11, wherein the in vivo blood glucose levels are regulated in a human.

15. A method of treating diabetes in a human or other mammal comprising administration to said human or other animal a combination comprising insulin and a peptide of the formula:
(Xaa)ₙ₁-Xaa₁-His-Thr-Asp-(Xaa)ₙ₂
wherein
Xaa is any amino acid;
Xaa₁ is a hydrophobic amino acid;
n₁ is 0-10; and
n₂ is 0-10 and derivatives thereof.

16. A method according to claim 15, wherein the peptide is one of the formulae:
(Xaa)ₙ₁-Val-His-Thr-Asp-(Xaa)ₙ₂; or
(Xaa)ₙ₁-Gly-His-Thr-Asp-(Xaa)ₙ₂;
wherein Xaa, n₁ and n₂ are as defined.

17. A method according to claim 15, wherein the peptide is a tetrapeptide selected from
Val-His-Thr-Asp (ISF402); and
Gly-His-Thr-Asp (ISF401);
or C-terminal and/or N-terminal capped derivatives thereof.

18. A method according to claim 15, wherein the diabetes is Type 1 diabetes.

19. A method according to claim 15, wherein the diabetes is Type 2 diabetes that requires administration of insulin.

20. A method according to claim 15, wherein the combination of insulin and peptide is administered in a single composition.

21. A method according to claim 15, wherein each component of the combination is administered separately, simultaneously or sequentially.

22. A method according to claim 15, wherein the combination is administered with another therapeutic agent.

23. A method according to claim 22, wherein the other therapeutic agent is another form of insulin or an insulin-sensitising agent.

24. A method of dispersing endogenous hexameric insulin complexes comprising the step of administering a peptide of the formula:
(Xaa)ₙ₁-Xaa₁-His-Thr-Asp-(Xaa)ₙ₂
wherein
Xaa is any amino acid;
Xaa₁ is a hydrophobic amino acid;
n₁ is 0-10; and
n₂ is 0-10 and derivatives thereof.
